(19) United States
(12) Patent Application Publication
(10) Pub. No.: US 2012/0251552 A1
(43) Pub. Date: Oct. 4, 2012

(54) METHODS OF GENERATING IMPROVED ANTIGEN-BINDING AGENTS USING CHAIN SHUFFLING AND OPTIONALLY SOMATIC HYPERMUTATION

(75) Inventor: Robert Horlick, San Diego, CA (US)

(73) Assignee: ANAPTYSBIO, INC., San Diego, CA (US)

(21) Appl. No.: 13/505,535

(22) PCT Filed: Nov. 3, 2010
(86) PCT No.: PCT/US10/55290

§ 371 Date: Jun. 7, 2012

Related U.S. Application Data
(60) Provisional application No. 61/258,457, filed on Nov. 5, 2009.

(51) Int. Cl. G01N 33/53; A61K 39/395; A61P 37/04; C07K 16/24

(52) U.S. Cl. 424/158.1; 435/7.1; 530/389.2

(57) ABSTRACT

The invention relates to a method of identifying a desired antigen-binding agent that binds to an antigen of interest. The method utilizes a combinatorial approach wherein a nucleic acid sequence encoding a polypeptide comprising a first component of an antigen-binding agent is provided to a population of cells together with a library of nucleic acid sequences, each of which encodes a polypeptide comprising a second component of an antigen-binding agent. The method further comprises subjecting one or more of the nucleic acid sequences encoding a first component, a second component, and/or an identified antigen-binding agent to somatic hypermutation.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 442,976 Byte ASCII (Text) file named “705436_ST25.TXT,” created on Nov. 4, 2009.

### BACKGROUND OF THE INVENTION

Natural mechanisms for generating antibody diversity exploit the process of somatic hypermutation (SHM) to trigger the evolution of immunoglobulin variable regions, thereby rapidly generating the secondary antibody repertoire associated with the humoral response. In vivo, SHM represents a highly efficient process, which is capable of rapidly exploring productive folding structures and evolving high affinity antibodies in a manner that represents the natural process for antibody optimization. Thus, there has been significant interest to try to replicate SHM in vitro to create a simple, robust process that would be capable of mimicking the natural processes of affinity maturation directly within a mammalian cellular context to select and evolve antibodies that are immunogenically tolerated, and highly expressed in mammalian cells (Cumbers et al., Nat. Biotechnol., 20(11): 1129-1134 (2002); Wang et al., Prot. Eng. Des. Sel., 17(9): 569-664 (2004); Wang et al., Proc. Natl. Acad. Sci. USA., 101(48): 16745-16749 (2004); Ruckerl et al., Mol. Immunol., 43 (10): 1645-1652 (2006); Todo et al., J. Biosci. Bioeng., 102(5): 478-81 (2006); Arakawa et al., Nucleic Acids Res., 36(1): e1 (2008)).

However, native antibodies that have been isolated from an individual human or animal often fail to demonstrate optimal affinity properties because an intrinsic affinity ceiling inherent in the immune system prevents the in vivo discrimination—and thus selection—of antibodies with affinities more potent than about 100 pM (Batista and Neuberger, Immunity, 8(6): 751-9 (1998) and EMBO J., 19(4): 513-20 (2000)).

The use of phage display libraries can address some of these issues, and phage display based approaches have been shown to be capable of routinely producing high affinity antibodies. For example, phage display has been used to increase the affinity of antibodies through an approach which has been described as “chain shuffling” (Marks, J. D., “Antibody Engineering: Methods and Protocols,” Methods in Molecular Biology, Vol. 248, Humana Press, NJ, pp. 327-343 (2004), and Kang et al., Proc. Natl. Acad. Sci. USA, 88: 11120-11123 (1991)). Phage display also has been used as a means for humanizing an antibody that binds to an antigen of interest (Jespers et al., Nat. Biotech., 12: 899-903 (1994); U.S. Pat. Nos. 5,565,332 and 6,258,562; U.S. Patent Application Publication 2006/0029594 A1; and International Patent Application Publication WO 93/06213).

From a theoretical perspective, however, such static phage display libraries are inherently limited in their size and scope, because even the largest (10¹²) libraries can explore only a small fraction of the potential innate immune repertoire. Additionally, the use of random mutagenesis in combination with phage display lacks the inherent selectivity profiling found in natural processes of antibody affinity maturation, often resulting in issues of human anti-human immunity, or undesirable cross reactivity profiles.

Chain shuffling on the surface of phage in bacterial systems as a means for identifying antibodies has additional inherent disadvantages due to the fact that most biologics are produced in mammalian systems. For example, it is not possible to simultaneously co-evolve antibodies via phage display approaches on the basis of both good mammalian expression and high affinity, which may lead to potential downstream manufacturing issues that result from otherwise poor expression in mammalian host cells. Moreover, bacteria lack key components of antibody production and processing, such as glycosylation and protein stabilization machinery, which are present in mammalian cells.

There remains a need for alternative and improved systems and methods to evolve high affinity antibodies directed to an antigen of interest. This invention provides such systems and methods. The invention further provides antibodies evolved by the systems and methods described herein as well as compositions comprising such antibodies and a carrier.

### BRIEF SUMMARY OF THE INVENTION

The invention provides a method of identifying an antigen-binding agent that binds to an antigen of interest. The method comprises (a) providing a population of cells containing a first nucleic acid sequence and a set of second nucleic acid sequences, wherein the first nucleic acid sequence encodes a first polypeptide comprising a first component of an antigen-binding agent, which first nucleic acid sequence optionally has been prepared by subjecting a nucleic acid sequence to somatic hypermutation, wherein the set of second nucleic acid sequences comprises second nucleic acid sequences encoding second polypeptides comprising second components of antigen-binding agents, which second nucleic acid sequences optionally have been prepared by subjecting a library of nucleic acid sequences to somatic hypermutation, and wherein (1) the first component and (2) a second component together form an antigen-binding agent, and wherein the population of cells optionally expresses activation-induced cytidine deaminase (AID), (b) maintaining the population of cells under conditions wherein the first nucleic acid sequence and the set of second nucleic acid sequences are expressed to produce a set of antigen-binding agents, (c) identifying an antigen-binding agent that binds to the antigen of interest, and (d) optionally subjecting one or both of the nucleic acid sequences encoding the identified antigen-binding agent to somatic hypermutation to provide mutant nucleic acid sequences encoding a desired antigen-binding agent that binds to the antigen of interest, with the proviso that at least one of the first nucleic acid sequence, the second nucleic acid sequences, and/or the nucleic acid sequences encoding the identified antigen-binding agent is subjected to somatic hypermutation.

The invention also provides a method of identifying an antigen-binding agent that binds to an antigen of interest comprising providing a population of mammalian cells containing a first nucleic acid sequence and a set of second nucleic acid sequences, wherein the first nucleic acid sequence encodes a first polypeptide comprising a first component of an antigen-binding agent, wherein the set of second nucleic acid sequences comprises second nucleic acid sequences encoding second polypeptides comprising second components of antigen-binding agents, and wherein (1) the first component and (2) a second component together form an antigen-binding agent, (b) maintaining the population of mammalian cells under conditions wherein the first nucleic acid sequence and the set of second nucleic acid sequences are expressed to produce a set of antigen-binding agents, and (c) identifying an antigen-binding agent that binds to the antigen of interest.

The invention further provides compositions comprising an antigen-binding agent and a carrier therefor, wherein the antigen-binding agent is provided by a method comprising: (a) providing a population of cells containing a first nucleic acid sequence and a set of second nucleic acid sequences, wherein the first nucleic acid sequence encodes a first polypeptide comprising a first component of an antigen-binding agent, which first nucleic acid sequence optionally has been prepared by subjecting a nucleic acid sequence to somatic hypermutation, wherein the set of second nucleic acid sequences comprises second nucleic acid sequences encoding second polypeptides comprising second components of antigen-binding agents, which second nucleic acid sequences optionally have been prepared by subjecting a library of nucleic acid sequences to somatic hypermutation, and wherein (1) the first component and (2) a second component together form an antigen-binding agent, and wherein the population of cells optionally expresses activation-induced cytidine deaminase (AID), (b) maintaining the population of cells under conditions wherein the first nucleic acid sequence and the set of second nucleic acid sequences are expressed to produce a set of antigen-binding agents, (c) identifying an antigen-binding agent that binds to the antigen of interest, and optionally, (d) subjecting one or both of the nucleic acid sequences encoding the identified antigen-binding agent to somatic hypermutation to provide mutant nucleic acid sequences encoding a desired antigen-binding agent that binds to the antigen of interest, with the proviso that at least one of the first nucleic acid sequence, the second nucleic acid sequences, and/or the nucleic acid sequences encoding the identified antigen-binding agent is subjected to somatic hypermutation.

### BRIEF DESCRIPTION OF THE DRAWINGS

FIGS. 1A-C are a set of flow cytometry plots of IgG expression (y-axis) and IL-17a-myc expression (x-axis) in HEK293 cells co-transfected with a fixed HC which had undergone somatic hypermutation (SHM) (hereinafter referred to as “mature HC”) and a library of kappa LCs. The cells were left untreated or were incubated with 25 nM of IL-17a-myc (round 1, FIG. 1A), 10 nM of IL-17a-myc (round 2, FIG. 1B), or 3 nM of IL-17a-myc (round 3, FIG. 1C). The cells screened in round 2 and round 3 were expanded from the positively gated cells collected in round 1 and round 2, respectively.

FIG. 2 is an alignment of the reference IL-17 antibody LC CDR3 and three sequence LOGOs. The sequence LOGOs illustrate the sequence conservation within each of the three LC sequence clades identified in round 1 of the screening depicted in FIG. 1. The arrows mark differences between clade consensus sequences.

FIG. 3 is a clade diagram of the LC sequences acquired from round 2 of the screening depicted in FIG. 1. The shaded ovals depict clades 2 and 3 identified in round 1 which are no longer represented in round 2. The asterisk indicates the sequence of the reference IL-17 antibody LC.

FIGS. 4A-C are a set of flow cytometry plots of IgG expression (y-axis) and IL-17a-myc expression (x-axis) in HEK293 cells co-transfected with a fixed HC, wherein all or part of the variable region, except the CDR3, was devoid of somatic hypermutation (SHM) (hereinafter referred to as “germline HC”) and a library of kappa LCs. The cells were left untreated or were incubated with 25 nM of IL-17a-myc antigen for each of the three screening rounds. The cells screened in round 2 (FIG. 4B) and round 3 (FIG. 4C) were expanded from the positively gated cells collected in round 1 (FIG. 4A) and round 2, respectively.

FIGS. 5A and B are a set of flow cytometry plots of IL-17a-myc expression (x-axis) in HEK293 cells co-transfected with a germline HC and a library of kappa LCs. The cells were left untreated or were incubated with 25 nM of IL-17a-myc antigen for both of the screening rounds. The cells screened in round 2 (FIG. 5B) were expanded from the positively gated cells collected in round 1 (FIG. 5A).

FIG. 6A is a clade diagram of LC sequences acquired from round 1 of the screening depicted in FIG. 4. The consensus sequences of the three identified clades are illustrated by sequence LOGOs. FIG. 6B are pie charts illustrating the relative frequencies of identifying a sequence falling within clade 1, 2, or 3 during round 1 and round 2 of screening with mature HC and round 1 of screening with germline HC.

### DETAILED DESCRIPTION OF THE INVENTION

The invention provides a method of identifying an antigen-binding agent that binds to an antigen of interest. The method comprises (a) providing a population of cells containing a first nucleic acid sequence and a set of second nucleic acid sequences, wherein the first nucleic acid sequence encodes a first polypeptide comprising a first component of an antigen-binding agent, which first nucleic acid sequence optionally has been prepared by subjecting a nucleic acid sequence to somatic hypermutation, wherein the set of second nucleic acid sequences comprises second nucleic acid sequences encoding second polypeptides comprising second components of antigen-binding agents, which second nucleic acid sequences optionally have been prepared by subjecting a library of nucleic acid sequences to somatic hypermutation, and wherein (1) the first component and (2) a second component together form an antigen-binding agent, and wherein the population of cells optionally expresses activation-induced cytidine deaminase (AID), (b) maintaining the population of cells under conditions wherein the first nucleic acid sequence and the set of second nucleic acid sequences are expressed to produce a set of antigen-binding agents, (c) identifying an antigen-binding agent that binds to the antigen of interest, and (d) optionally subjecting one or both of the nucleic acid sequences encoding the identified antigen-binding agent to somatic hypermutation to provide mutant nucleic acid sequences encoding a desired antigen-binding agent that binds to the antigen of interest, with the proviso that at least one of the first nucleic acid sequence, the second nucleic acid sequences, and/or the nucleic acid sequences encoding the identified antigen-binding agent is subjected to somatic hypermutation.

In some embodiments, the first nucleic acid sequence has not been prepared by subjecting a nucleic acid sequence to somatic hypermutation, and the second nucleic acid sequences have been prepared by subjecting a library of nucleic acid sequences to somatic hypermutation.

In other embodiments, the first nucleic acid sequence has been prepared by subjecting a nucleic acid sequence to somatic hypermutation, and the second nucleic acid sequences have not been prepared by subjecting a library of nucleic acid sequences to somatic hypermutation.

In yet other embodiments, the first nucleic acid sequence has been prepared by subjecting a nucleic acid sequence to somatic hypermutation, and the second nucleic acid sequences have been prepared by subjecting a library of nucleic acid sequences to somatic hypermutation.

In still other embodiments, the first nucleic acid sequence has not been prepared by subjecting a nucleic acid sequence to somatic hypermutation, and the second nucleic acid sequences have not been prepared by subjecting a library of nucleic acid sequences to somatic hypermutation.

The invention also encompasses utilizing one or more of the mutant nucleic acid sequences obtained by the inventive method to provide a desired antigen-binding agent that binds to an antigen of interest. In certain embodiments, the method comprises repeating steps (a), (b), (c), and optionally (d) of the inventive method, except that, in the repeated steps, the first nucleic acid sequence comprises one of the mutant nucleic acid sequences encoding the desired antigen-binding agent provided by initial steps (a), (b), (c), and optionally (d).

An “antigen” is a molecule that induces an immune response in a mammal. An “immune response” can entail, for example, antibody production and/or the activation of immune effector cells (e.g., T-cells). An antigen in the context of the invention can comprise any subunit, fragment, or epitope of any proteinaceous or non-proteinaceous (e.g., carbohydrate or lipid) molecule which provokes an immune response in mammal. By “epitope” is meant a sequence on an antigen that is recognized by an antibody or an antigen receptor. Epitopes also are referred to in the art as “antigenic determinants.”

By “antigen-binding agent” is meant a molecule, preferably a proteinaceous molecule, that specifically binds to an antigen. The antigen-binding agent comprises at least two components (i.e., the aforementioned first and second components) which, in combination (or together), form the antigen-binding site of an antigen-binding agent. The antigen-binding agent also can consist of the two components, i.e., the aforementioned first and second components, which, in combination (or together) without other components, form the antigen-binding agent. It is not a requirement that both components are directly involved in antigen-binding. For example, the second component may contribute to a conformational change of the antigen-binding agent that facilitates antigen binding but which does not in itself directly bind to the antigen. Furthermore, it is not a requirement that the first and second components are separate polypeptides. For example, the first and second components can be contained within a single polypeptide chain, optionally separated by a linker. The antigen-binding agent can have one antigen-binding site or more than one (e.g., two or more) antigen binding sites. For example, the antigen-binding agent may bind to a second antigen on a molecule that is separate and distinct from the antigen of interest, or to a second epitope on the antigen of interest.

Preferably, the antigen-binding agent is an antibody or a fragment (e.g., immunogenic fragment) thereof. The antigen-binding agent can be a whole antibody. A whole antibody consists of four polypeptides: two identical copies of a heavy (H) chain and two copies of a light (L) chain. Each of the heavy chains contains one N-terminal variable (V_{H}) region and three C-terminal constant (CH₁, CH₂ and CH₃) regions, and each light chain contains one N-terminal variable (V_{L}) region and one C-terminal constant (CO region. The light chains of antibodies can be assigned to one of two distinct types, either kappa (K) or lambda (λ), based upon the amino acid sequences of their constant domains. In a typical whole antibody, each light chain is linked to a heavy chain by disulphide bonds, and the two heavy chains are linked to each other by disulphide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen-binding site of an antibody. The V_{H} and V_{L} regions have the same general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding. The four framework regions (FWs or FRs) largely adopt a beta-sheet conformation, and the CDRs form loops connecting, and in some cases comprising part of, the beta-sheet structure.

The constant regions of the light and heavy chains are not directly involved in binding of the antibody to an antigen, but exhibit various effector functions, such as participation in antibody-dependent cellular toxicity via interactions with effector molecules and cells.

The antigen-binding agent can also be a fragment of an antibody. The terms “fragment of an antibody,” “antibody fragment,” or “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). Examples of antibody fragments include but are not limited to (i) a Fab fragment, which is a monovalent fragment consisting of the V_{L}, V_{H}, C_{L}, and C_{H}1 domains; (ii) a F(ab′)₂ fragment, which is a bivalent fragment comprising two Fab fragments linked by a disulfide bridge at the hinge region; and (iii) a Fv fragment consisting of the V_{L} and V_{H} domains of a single arm of an antibody.

The antigen-binding agent can also be a single chain antibody fragment. Examples of single chain antibody fragments include but are not limited to (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); and Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen-binding sites. Antibody fragments are known in the art and are described in more detail in, e.g., U.S. Patent Application Publication 2009/0093024 A1.

The antigen-binding agent can also be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

In one embodiment, the first polypeptide comprising a first component of an antigen-binding agent is an antibody heavy chain or a fragment thereof, and each of the second polypeptides comprising second components of antigen-binding agents is an antibody light chain or fragment thereof. In another embodiment, the first polypeptide comprising a first component of an antigen-binding agent is an antibody light chain or a fragment thereof, and each of the second polypeptides comprising second components of antigen-binding agents is an antibody heavy chain or fragment thereof. The antibody light chain can be a kappa (κ) or lambda (λ) light chain. Preferably, the light chain is a kappa light chain.

Any antigen-binding fragment of an antibody heavy chain can be used in the context of the invention. The fragment desirably comprises, for example, one or more CDRs, the variable region (or portions thereof), the constant region (or portions thereof), or combinations thereof. Likewise, any fragment of an antibody light chain can be used in the invention. The fragment of an antibody light chain desirably comprises, for example, one or more CDRs, the variable region, the constant region, or combinations thereof. The fragment can be any portion of a heavy chain or a light chain provided that, in combination, the first component and the second component form an antigen-binding agent.

The first component of an antigen-binding agent can be obtained from a human antibody, a non-human antibody, or a chimeric antibody. By “chimeric” is meant an antibody or fragment thereof comprising both human and non-human regions. Non-human antibodies include antibodies isolated from any non-human animal, such as, for example, a rodent (e.g., a mouse or rat). The second component of an antigen-binding agent also can be obtained from a human antibody, a non-human antibody, or a chimeric antibody, independent of whether the first component of an antigen-binding agent is obtained from a human antibody, a non-human antibody, or a chimeric antibody. In other words, for example, the first component can be obtained from a human antibody, and the second component can be obtained from a non-human antibody. Conversely, the first component can be obtained from a non-human antibody, and the second component can be obtained from a human antibody. For example, the first component can be a nucleic acid encoding a polypeptide comprising a rodent antibody or fragment thereof, and the second component can be a library of nucleic acids, each of which encodes a polypeptide comprising a human antibody or fragment thereof. This scenario may be useful, e.g., for the humanization of an antigen-binding agent. In another embodiment, the first component and the second component are both obtained from a human antibody or a non-human antibody. Alternatively, the first component and the second component are both chimeric antibodies.

A human antibody, a non-human antibody, or a chimeric antibody can be obtained by any means, including in vitro sources (e.g., a hybridoma or a cell line producing an antibody recombinantly) and in vivo sources (e.g., rodents). Methods for generating antibodies are known in the art and are described in, for example, see, e.g., Köhler and Milstein, Eur. J. Immunol., 5: 511-519 (1976), Harlow and Lane (eds.), Antibodies: A Laboratory Manual, CSH Press (1988), and C. A. Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the HUMAB-MOUSE®, the Kirin TC MOUSE™, and the KM-MOUSE® (see, e.g., Lonberg N., Nat. Biotechnol., 23(9): 1117-25 (2005), and Lonberg N., Handb. Exp. Pharmacol., 181: 69-97 (2008)).

Instead of an antibody or a fragment thereof, the antigen-binding agent also can be an “alternative scaffold” or a fragment thereof. By “alternative scaffold” is meant a non-antibody polypeptide or polypeptide domain which displays and affinity and specificity towards an antigen of interest similar to that of an antibody. Exemplary alternative scaffolds include a β-sandwich domain such as from fibronectin (e.g., Adnectins), lipocalins (e.g., Anticalin), a Kunitz domain, thioredoxin (e.g., peptide aptamer), protein A (e.g., AFFIBODY® molecules), an ankyrin repeat (e.g., DARPins), γ-β-crystallin or ubiquitin (e.g., AFFLIN™ molecules), CTLD₃ (e.g., Tetranectin), and multivalent complexes (e.g., ATRIMER™ molecules or SIMP™ molecules). Alternative scaffolds are described in, for example, Binz et al., Nat. Biotechnol., 23: 1257-1268 (2005); Skerra, Curr. Opin. Biotech., 18: 295-304 (2007); and U.S. Patent Application Publication 2009/0181855 A1.

In one embodiment, the alternative scaffold can be an AVIMER™ molecule. An AVIMER™ molecule is a class of therapeutic proteins from human origin unrelated to antibodies and antibody fragments, which are composed of several modular and reusable binding domains, referred to as A-domains (also referred to as class A module, complement type repeat, or LDL-receptor class A domain). AVIMER™ molecules were developed from human extracellular receptor domains by in vitro exon shuffling and phage display (Silverman et al., Nat. Biotechnol., 23: 1493-94 (2005), and Silverman et al., Nat. Biotechnol. 24: 220 (2006)). AVIMER™ molecules may comprise multiple independent binding domains that may exhibit improved affinity (in some cases sub-nanomolar) and specificity compared with single-epitope binding proteins. See, e.g., U.S. Patent Application Publications 2005/0221384 A1; 2005/0164301 A1; 2005/0053973 A1; 2005/0089932 A1; 2005/0048512 A1; and 2004/0175756 A1. Each of the known 217 human A-domains comprises about 35 amino acids (about 4 kDa). Native A-domains fold quickly and efficiently to a uniform, stable structure mediated primarily by calcium binding and disulfide formation. A conserved scaffold motif of only twelve amino acids is required for this common structure. An AVIMER™ molecule comprises multiple A-domains which are separated from one another by linkers that average five amino acids in length. The end result is a single protein chain containing multiple domains, each of which represents a separate function. Each domain of an AVIMER™ molecule binds an antigen independently, and the energetic contributions of each domain are additive.

The antigen-binding agent can also be a conjugate of (1) an antibody, an alternative scaffold, or fragments thereof, and (2) a protein or non-protein moiety. For example, the antigen-binding agent can be an antibody conjugated to a peptide, a fluorescent molecule, or a chemotherapeutic agent.

In the context of the invention, the first component and second component of an antigen-binding agent are typically derived from an antibody that binds an antigen of interest. A polypeptide comprising the first component of an antigen-binding agent is provided to a population of cells in the form of a nucleic acid encoding the polypeptide. A “nucleic acid” is intended to encompass a polymer of DNA or RNA, i.e., a polynucleotide, which can be single-stranded or double-stranded and which can contain non-natural or altered nucleotides. Nucleic acids are typically linked via phosphate bonds to form nucleic acids or polynucleotides, though many other linkages are known in the art (e.g., phosphorothioates, boranophosphates, and the like).

If the nucleic acid sequence encoding a polypeptide comprising a first or second component of an antigen-binding agent is not known, it can be determined by cloning the nucleic acid from a source which expresses the antibody that binds an antigen of interest, such as a hybridoma, by standard molecular biology techniques (see, e.g., Sambrook et al, Molecular Cloning: A Laboratory Manual, second edition, Cold Spring Harbor Laboratory, (1989)).

In some embodiments, a first and/or one or more second component of an antigen-binding agent can be “devolved.” By “devolved” is meant that the polypeptide comprising a component of an antigen-binding agent has undergone one or more amino acid modifications so as to reduce its affinity or avidity for binding to antigen. The process of generating a devolved component of an antigen-binding agent can be achieved, for example, by reverting the amino acid sequence of the antigen-binding region of an antibody which has undergone one or more somatic hypermutation events to its germline equivalent.

As discussed herein, the first and second components need not be provided on separate polypeptides. For example, the first and second components can be contained within a single polypeptide chain, optionally separated by a linker. In this regard, the first nucleic acid sequence and at least one of the second nucleic acid sequences can be provided on the same nucleic acid molecule. Alternatively, the first nucleic acid sequence and at least one of the second nucleic acid sequences can be provided on different nucleic acid molecules. The first nucleic acid sequence and the set of second nucleic acid sequences can be provided to cells simultaneously or sequentially.

The second component of the antigen-binding agent preferably is provided to a population of cells in the form of a library comprising second nucleic acid sequences encoding second polypeptides comprising second components of antigen-binding agents. As used herein, the term “library” refers to a plurality of polynucleotides, proteins, or cells comprising a collection of two or more (e.g., 10 or more, 100 or more, 1,000 or more, 10,000 or more, or 100,000 or more) non-identical members. In accordance with the invention, the library can comprise a mixture of human and non-human polynucleotide or gene sequences. The library can be generated by enzymatic amplification (e.g., PCR) of human or non-human cells or tissues.

In certain embodiments, the library is a “synthetic library.” By “synthetic library” is meant a plurality of “synthetic polynucleotides” or “synthetic genes” or a population of cells that comprises a plurality of “synthetic polynucleotides” or “synthetic genes.” By “synthetic polynucleotide” or “synthetic gene” is meant that the corresponding polynucleotide sequence or portion thereof is derived from a sequence that has been designed, or synthesized de-novo, or modified, compared to the equivalent naturally occurring sequence. Synthetic polynucleotides or synthetic genes can be prepared by methods known in the art, including but not limited to, chemical synthesis or via PCR amplification (or similar enzymatic amplification systems).

In other embodiments, the library is a “semi-synthetic library.” By “semi-synthetic library” is meant a plurality of “semi-synthetic polynucleotides” or “semi-synthetic genes” or a population of cells that comprises a plurality of “semi-synthetic polynucleotides” or “semi-synthetic genes.” The terms “semi-synthetic polynucleotide” and “semi-synthetic gene” refer to polynucleotide sequences that consist in part of a nucleic acid sequence that has been obtained via polymerase chain reaction (PCR) or other similar enzymatic amplification system which utilizes a natural donor (e.g., peripheral blood monocytes) as the starting material for the amplification reaction. The remaining “synthetic” polynucleotides, i.e., those portions of semi-synthetic polynucleotide not obtained via PCR or other similar enzymatic amplification system, can be synthesized de novo using methods known in the art including, but not limited to, the chemical synthesis of nucleic acid sequences.

A synthetic library or a semi-synthetic library can comprise polynucleotides or genes which have undergone one or more somatic hypermutation (SHM) events. In other embodiments, the library can be a “seed library.” By “seed library” is meant a plurality (e.g., 2 or more, 10 or more, 100 or more, 1,000 or more, 10,000 or more, or 100,000 or more) of synthetic or semi-synthetic polynucleotides, or cells that comprise such polynucleotides, that contain one or more sequences or portions thereof which have been modified to act as a substrate for somatic hypermutation (SHM) and which are capable, when acted upon by SHM, to create a library of polynucleotides, proteins, or cells in situ. Methods of generating libraries are described in, for example, U.S. Patent Application Publication 2009/0093024 A1.

The antigen-binding agent can be identified using a variety of methods known in the art. Preferably, the antigen-binding agent is identified using a method known in the art as “chain shuffling.” Chain shuffling (also referred to in the art as “guided selection” or “combinatorial antibody libraries”) involves identifying an antibody that binds to an antigen of interest from, e.g., a phage display library. The nucleic acid sequence encoding a component of the antigen binding site of the antibody (e.g., the light chain variable region) is then diversified by random- or site-specific-mutation, while the nucleic acid sequence encoding another component of the antigen binding site of the antibody (e.g., the heavy chain variable region) is fixed. This can be accomplished, for example, by cloning a wild-type nucleic acid sequence encoding a heavy chain variable region of an antibody which binds an antigen of interest into a phage display vector system which contains a library of diversified light chain variable region nucleic acid sequences and screening for high-affinity binders on antigen (Marks, supra). Typically, the light chain variable region is shuffled first while the heavy chain variable region is fixed, since the latter contains most of the binding energy of an antibody-antigen interaction, although shuffling of the heavy chain variable region with a fixed light chain variable region has also been described (Kang et al., Proc. Natl. Acad. Sci. USA, 88: 11120-11123 (1991)).

Chain shuffling on the surface of phage has also been used as a means for humanizing an antibody that binds to an antigen of interest (Jespers et al., Nat. Biotech., 12: 899-903 (1994)). In this approach, a first component of the antigen binding site of a non-human antibody (e.g., the heavy chain variable region of a rodent antibody) is co-displayed as a Fab fragment on the surface of phage displaying a library of a second component of the antigen binding site of a human antibody (e.g., a library of human κ light chains), and the phage are screened for binding to antigen (Jespers et al., supra). Subsequently, the second component of the antigen binding site of a phage which bound the antigen is co-displayed as a Fab fragment on the surface of phage displaying a library of a first component of the antigen binding site of a human antibody (e.g., a library of human heavy chains), and the phage are again screened for binding to antigen. This process, often referred to as “guided selection,” has been used, for example, to generate a fully humanized antibody that binds to TNFα utilizing a mouse antibody that binds to TNFα for guidance (Jespers et al., supra). The generation of humanized antibodies by chain shuffling using rodent antibodies as templates is described in, e.g., U.S. Pat. Nos. 5,565,332 and 6,258,562, U.S. Patent Application Publication 2006/0029594 A1, and International Patent Application Publication WO 93/06213.

In certain embodiments of the invention, at least one of the first nucleic acid sequence, the second nucleic acid sequences, and/or the nucleic acid sequences encoding the identified antigen-binding agent is subjected to somatic hypermutation (SHM). In other words, SHM can occur before, during, or after identification of the antigen-binding agent (e.g., via chain-shuffling). In some embodiments, the first nucleic acid sequence and/or the set of second nucleic acid sequences are subjected to SHM prior to the identification of an antigen-binding agent that binds to the antigen of interest. In other embodiments, the first nucleic acid sequence and/or the set of second nucleic acid sequences are subjected to SHM concomitantly with identification of the antigen-binding agent (e.g., by way of expressing activiation-induced cytidine deaminase (AID) in the population of cells). Alternatively, one or both of the nucleic acid sequences encoding the identified antigen-binding agent are subjected to SHM (e.g., after chain shuffling). SHM can occur at multiple time points in accordance with the methods of the invention. The timing of the induction of SHM can be determined by one of ordinary skill in the art in accordance with the desired characteristics of an antigen-binding agent.

As used herein, “somatic hypermutation” or “SHM” refers to the mutation of a polynucleotide sequence which can be initiated by, or associated with, the action of activation-induced cytidine deaminase (AID), which includes members of the AID/APOBEC family of RNA/DNA editing cytidine deaminases that are capable of mediating the deamination of cytosine to uracil within a DNA sequence (see, e.g., Conticello et al., Mol. Biol. Evol., 22: 367-377 (2005), and U.S. Pat. No. 6,815,194). SHM can also be initiated by, or associated with the action of, e.g., uracil glycosylase and/or error prone polymerases on a polynucleotide sequence of interest. SHM is intended to include mutagenesis that occurs as a consequence of the error prone repair of an initial DNA lesion, including mutagenesis mediated by the mismatch repair machinery and related enzymes. The term “substrate for SHM” refers to a synthetic or semi-synthetic polynucleotide sequence which is acted upon by AID and/or error prone DNA polymerases to effect a change (i.e., a mutation) in the nucleic acid sequence of the synthetic or semi-synthetic polynucleotide sequence.

In certain embodiments of the invention, AID can be endogenous to the cells expressing one or more components of an antigen-binding agent. Alternatively, a nucleic acid encoding AID may be provided to cells which do, or which do not, contain an endogenous AID protein. The exogenously provided AID can be a wild-type AID, which refers to a naturally occurring amino acid sequence of an AID protein. Suitable wild-type AID proteins include all vertebrate forms of AID, including, for example, primate, rodent, avian, and bony fish. Representative examples of wild-type AID amino acid sequences include without limitation, human AID (SEQ ID NO: 1 or SEQ ID NO: 2), canine AID (SEQ ID NO: 3), murine AID (SEQ ID NO: 4), rat AID (SEQ ID NO: 5), bovine AID (SEQ ID NO: 6), chicken AID (SEQ ID NO: 7), porcine AID (SEQ ID NO: 8), chimp AID (SEQ ID NO: 9), macaque AID (SEQ ID NO: 10), horse AID (SEQ ID NO: 11), Xenopus AID (SEQ ID NO: 12), pufferfish (fugu) AID (SEQ ID NO: 13), and zebrafish (SEQ ID NO: 14). The use of AID in SHM systems is described in detail in, for example, U.S. Patent Application Publication 2009/0075378 A1 and International Patent Application Publications WO 08/103,474 and WO 08/103,475.

In other embodiments, the exogenously provided AID can be a an “AID mutant” or a “mutant of AID.” As used herein, an “AID mutant” or a “mutant of AID” refers to an AID amino acid sequence that differs from a wild-type AID amino acid sequence by at least one amino acid. A wild-type amino acid sequence can be mutated to produce an AID mutant by any suitable method known in the art, such as, for example, by insertion, deletion, and/or substitution. For example, mutations may be introduced into a nucleic acid sequence encoding wild-type AID randomly or in a site-specific manner. Random mutations may be generated, for example, by error-prone PCR of an AID template sequence. A preferred means for introducing random mutations in is the Genemorph II Random Mutagenesis Kit (Stratagene, LaJolla, Calif.). Site-specific mutations can be introduced, for example, by ligating into an expression vector a synthesized oligonucleotide comprising the modified site. Alternately, oligonucleotide-directed site-specific mutagenesis procedures can be used, such as those disclosed in Walder et al., Gene, 42: 133 (1986); Bauer et al., Gene, 37: 73 (1985); Craik, Biotechniques, 12-19 (January 1995); and U.S. Pat. Nos. 4,518,584 and 4,737,462. A preferred means for introducing site-specific mutations is the QuikChange Site-Directed Mutagenesis Kit (Stratagene, LaJolla, Calif.).

Preferably, an AID mutant is a “functional mutant of AID” or a “functional AID mutant,” which refers to a mutant AID protein which retains all or part of the biological activity of a wild-type AID, or which exhibits increased biological activity as compared to a wild-type AID protein. The biological activity of a wild-type AID includes, but is not limited to, the deamination of cytosine to uracil within a DNA sequence, papillation in a bacterial mutagenesis assay, somatic hypermutation of a target gene, and immunoglobulin class switching. A mutant AID protein can retain any part of the biological activity of a wild-type AID protein. Desirably, the mutant AID protein retains at least 75% (e.g., 75% or more, 80% or more, or 90% or more) of the biological activity of wild-type AID. Preferably, the mutant AID protein retains at least 90% (e.g., 90% or more, 95% or more, or 100% or more) of the biological activity of wild-type AID.

In a preferred embodiment, the mutant AID protein exhibits increased biological activity as compared to a wild-type AID protein. In this respect, the functional AID mutant can display at least a 10-fold improvement in activity compared to a wild-type AID protein as measured, e.g., by a bacterial papillation assay (see, e.g., Nghiem et al., Proc. Natl. Acad. Sci. USA, 85: 2709-2713 (1988), and Ruiz et al., J. Bacteriol., 175: 4985-4989 (1993)). Suitable mutant AID proteins which exhibit increased biological activity as compared to a wild-type AID protein are described in Wang et al., Nat. Struct. Mol. Biol., 16(7): 769-76 (2009), and U.S. Provisional Patent Application No. 61/166,349.

In still other embodiments, SHM can be initiated by, or associated with the action of, an “AID homolog.” The term “AID homolog” refers to the enzymes of the Apobec family and include, for example, Apobec-1, Apobec3C, or Apobec3G (described, for example, in Jarmuz et al., Genomics, 79: 285-296 (2002)). The term “AID activity” includes activity mediated by AID and AID homologs.

In certain embodiments of the invention, the first nucleic acid sequence has been modified as compared to a corresponding wild-type nucleic acid sequence to increase or decrease the density of SHM cold spots and/or SHM hot spots so as to increase or decrease the susceptibility of the nucleic acid sequence to SHM. In addition, one or more of the second nucleic acid sequences can be modified as compared to a corresponding wild-type nucleic acid sequence to increase or decrease the density of SHM cold spots and/or SHM hot spots so as to increase or decrease the susceptibility of the nucleic acid sequence to SHM. In one embodiment, both the first nucleic acid sequence and one or more of the second nucleic acid sequences of the library have been modified as compared to the corresponding wild-type nucleic acid sequences to increase or decrease the density of SHM cold spots and/or SHM hot spots so as to increase or decrease the susceptibility of the nucleic acid sequences to SHM.

As used herein, the term “SHM hot spot” or “hot spot” refers to a polynucleotide sequence, or motif, of 3-6 nucleotides that exhibits an increased tendency to undergo somatic hypermutation, as determined via a statistical analysis of SHM mutations in antibody genes. Likewise, as used herein, a “SHM cold spot” or “cold spot” refers to a polynucleotide sequence, or motif, of 3-6 nucleotides that exhibits a decreased tendency to undergo somatic hypermutation, as determined via a statistical analysis of SHM mutations in antibody genes. A relative ranking of various motifs for SHM as well as canonical hot spots and cold spots in antibody genes are described in U.S. Patent Application Publication 2009/0075378 A1 and International Patent Application Publication WO 08/103,475, and the statistical analysis can be extrapolated to an analysis of SHM mutations in non-antibody genes as described therein.

The term “somatic hypermutation motif” or “SHM motif” refers to a polynucleotide sequence that includes, or can be altered to include, one or more hot spots or cold spots, and which encodes a defined set of amino acids. SHM motifs can be of any size, but typically are from about 2 to about 20 nucleotides in size, e.g., from about 3 to about 9 nucleotides in size. SHM motifs can include any combination of hot spots and cold spots, or may lack both hot spots and cold spots.

The terms “preferred hot spot SHM codon,” “preferred hot spot SHM motif,” “preferred SHM hot spot codon,” and “preferred SHM hot spot motif” refer to a codon including, but not limited to, codons AAC, TAC, TAT, AGT, or AGC. Such sequences may be potentially embedded within the context of a larger SHM motif, recruit SHM mediated mutagenesis, and generate targeted amino acid diversity at that codon.

As used herein, a nucleic acid sequence has been “optimized for SHM” if the nucleic acid sequence, or a portion thereof, has been altered to increase or decrease the frequency and/or location of hot spots and/or cold spots within the nucleic acid sequence. A nucleic acid sequence has been made “susceptible to SHM” if the nucleic acid sequence, or a portion thereof, has been altered to increase the frequency and/or location of hot spots within the nucleic acid sequence or to decrease the frequency (density) and/or location of cold spots within the nucleic acid sequence as compared to a corresponding wild-type nucleic acid sequence. Conversely, a nucleic acid sequence has been made “resistant to SHM” if the nucleic acid sequence, or a portion thereof, has been altered to decrease the frequency (density) and/or location of hot spots within the open reading frame of the nucleic acid sequence or to increase the frequency and/or location of cold spots within the nucleic acid sequence as compared to a corresponding wild-type nucleic acid sequence. In general, a nucleic acid sequence can be prepared that has a greater or lesser propensity to undergo SHM mediated mutagenesis by altering the codon usage, and/or the amino acids encoded by the nucleic acid sequence, as described in, for example, U.S. Patent Application Publication 2009/0075378 A1 and International Patent Application Publication WO 08/103,475.

Optimization of a nucleic acid sequence refers to modifying about 1%, about 2%, about 3%, about 4%, about 5%, about 10%, about 20%, about 25%, about 50%, about 75%, about 90%, about 95%, about 96%, about 97%, about 98%, about 99%, about 100%, or a range defined by any two of the foregoing values, of the nucleotides in the nucleic acid sequence. Optimization of a nucleic acid sequence also refers to modifying about 1, about 2, about 3, about 4, about 5, about 10, about 20, about 25, about 50, about 75, about 90, about 95, about 96, about 97, about 98, about 99, about 100, about 200, about 300, about 400, about 500, about 750, about 1000, about 1500, about 2000, about 2500, about 3000 or more, or a range defined by any two of the foregoing values, of the nucleotides in the nucleic acid sequence such that some or all of the nucleotides are optimized for SHM-mediated mutagenesis. Reduction in the frequency (i.e., density) of hot spots and/or cold spots refers to reducing the number of hot spots and/or cold spots in a nucleic acid sequence by about 1%, by about 2%, by about 3%, by about 4%, by about 5%, by about 10%, by about 20%, by about 25%, by about 50%, by about 75%, by about 90%, by about 95%, by about 96%, by about 97%, by about 98%, by about 99%, by about 100%, or a range defined by any two of the foregoing values. Increasing the frequency (i.e., density) of hot spots and/or cold spots refers to increasing the number of hot spots and/or cold spots in a nucleic acid sequence by about 1%, by about 2%, by about 3%, by about 4%, by about 5%, by about 10%, by about 20%, by about 25%, by about 50%, by about 75%, by about 90%, by about 95%, by about 96%, by about 97%, by about 98%, by about 99%, by about 100%, or a range defined by any two of the foregoing values.

The position or reading frame of a hot spot or cold spot is also a factor affecting whether SHM-mediated mutagenesis results in a mutation that is silent with regards to the resulting amino acid sequence, or causes conservative, semi-conservative, or non-conservative changes at the amino acid level. The design parameters can be manipulated to further enhance the relative susceptibility or resistance of a nucleotide sequence to SHM. Thus, both the degree of SHM recruitment and the reading frame of the motif are considered in the design of SHM susceptible and SHM resistant nucleic acid sequences.

In a preferred embodiment of the invention, the nucleic acid sequence that is subjected to SHM encodes a polypeptide comprising a first or a second component of an antigen-binding agent which is an antibody, or a portion thereof that can bind to an antigen. Nucleic acid sequences which may be subjected to SHM in accordance with the invention include nucleic acid sequences encoding, e.g., naturally occurring germline, affinity matured, synthetic, or semi-synthetic antibodies. Nucleic acid sequences encoding antibody fragments, such as, e.g., F(ab′)2, Fab′, Fab, Fv, scFv, dsFv, dAb, or a single chain binding polypeptide, also may be subjected to SHM. In general, such antibody-encoding sequences can be altered through SHM to improve one or more of the following functional traits: affinity, avidity, selectivity, stability (including but not limited to thermal stability and proteolytic stability), solubility, folding, immunotoxicity, expression, and catalytic activity. The inventive method also can be used to generate antibodies which have been subjected to SHM in the constant domain (e.g., Fc), which can result in increased binding affinity for an Fc receptor (FcR), thereby modulating signal cascades.

It will be appreciated that there are a variety of other nucleic acid sequences, such as coding sequences and genetic elements, that one of ordinary skill in the art would prefer to not undergo SHM in order to maintain overall system integrity. Examples of such nucleic acid sequences include (i) selectable markers, (ii) reporter genes, (iii) genetic regulatory signals, (iv) enzymes or accessory factors used for high level enhanced SHM, or its regulation or measurement (e.g., AID or a functional AID mutant, pol eta, transcription factors, and MSH2), (v) signal transduction components (e.g., kinases, receptors, transcription factors), and (vi) domains or sub domains of proteins (e.g., nuclear localization signals, transmembrane domains, catalytic domains, protein-protein interaction domains, and other protein family conserved motifs, domains, and sub-domains).

Depending on the nature of the antigen-binding agent of interest, and the amount of information available on the antigen-binding agent of interest, one of ordinary skill in the art can follow any combination of the following strategies prior to, or in conjunction with, practicing the inventive method to identify an antigen-binding agent with a desired property: (i) no SHM optimization, (ii) global optimization, and (iii) selective SHM hot spot modification.

Although it can be desirable to enhance the number of hot spots within the nucleic acid sequence encoding a component of an antigen-binding agent of interest, it should be noted that any unmodified nucleic acid sequence is expected to undergo a certain amount of SHM, and can be used in the inventive method without optimization, or any specific knowledge of the actual sequence. Moreover, certain proteins (e.g., antibodies) naturally comprise nucleic acid sequences which have evolved suitable codon usage, and do not require codon modification. Alternatively, it can be desirable to enhance the number of cold spots within the nucleic acid sequence encoding a component of an antigen-binding agent of interest (e.g., framework regions of antibodies or fragments thereof).

In some aspects, the number of hotspots in a nucleic acid sequence encoding a component of an antigen-binding agent of interest can be increased, as described in detail in, for example, U.S. Patent Application Publication 2009/0075378 A1 and International Patent Application Publication WO 08/103,475. This approach can be applied to the entire coding region of the nucleic acid sequence, thereby rendering the entire nucleic acid sequence more susceptible to SHM. This approach can be preferred if relatively little is known about structure activity relationships of the antigen-binding agent.

Alternatively, a nucleic acid sequence encoding a component of an antigen-binding agent of interest can be selectively and/or systematically modified through the targeted replacement of regions of interest with synthetic variable regions as described in, for example, U.S. Patent Application Publication 2009/0075378 A1 and International Patent Application Publication WO 08/103,475, which provide for a high density of hot spots and seed maximal diversity through SHM at specific loci. One of ordinary skill in the art would understand, based on the foregoing, that any or all of the above approaches can be undertaken in conjunction with the inventive method.

Following the design of a nucleic acid sequence that has been modified to increase or decrease the susceptibility of the nucleic acid sequence to SHM, the nucleic acid sequence can be synthesized using standard methodologies and sequenced to confirm correct synthesis.

The nucleic acids encoding polypeptides comprising the first and second components of an antigen-binding agent typically are provided to a population of cells in the form of a vector, such as a plasmid, episome, cosmid, viral vector (e.g., retroviral or adenoviral), or phage. Suitable vectors and methods of vector preparation are well known in the art (see, e.g., Sambrook et al., supra, and Maniatis et al., Cell Biology: A Comprehensive Treatise, Vol. 3, Gene Sequence Expression, Academic Press, NY, pp. 563-608 (1980)). Preferably, the vector is a replicable genetic display package. By “replicable genetic display package” is meant a biological particle comprising genetic information which provides the particle with the ability to replicate. The particle can display on its surface at least part of a polypeptide comprising a first and/or a second component of an antigen-binding agent. The polypeptide(s) can be encoded by genetic information native to the particle and/or inserted recombinantly into the particle or an ancestor of the particle. In certain embodiments, the replicable genetic display package is a viral vector or a bacteriophage.

In addition to the nucleic acid encoding a polypeptide comprising a first or a second component of an antigen-binding agent, the vector preferably comprises expression control sequences, such as promoters, enhancers, polyadenylation signals, transcription terminators, internal ribosome entry sites (IRES), and the like, that provide for the expression of the coding sequence in a host cell. Exemplary expression control sequences are known in the art and described in, for example, Goeddel, Gene Expression Technology: Methods in Enzymology, Vol. 185, Academic Press, San Diego, Calif. (1990).

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include, for example, the Tet system (U.S. Pat. Nos. 5,464,758 and 5,814,618), the Ecdysone inducible system (No et al., Proc. Natl. Acad. Sci., 93: 3346-3351 (1996)), the T-REx™ system (Invitrogen, Carlsbad, Calif.), LacSwitch® System (Stratagene, San Diego, Calif.), and the Cre-ERT tamoxifen inducible recombinase system (Indra et al., Nuc. Acid. Res., 27: 4324-4327 (1999); Nuc. Acid. Res., 28: e99 (2000); U.S. Pat. No. 7,112,715; and Kramer & Fussenegger, Methods Mol. Biol., 308: 123-144 (2005)).

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. The term “Ig enhancers” refers to enhancer elements derived from enhancer regions mapped within the immunoglobulin (Ig) locus (such enhancers include for example, the heavy chain (mu) 5′ enhancers, light chain (kappa) 5′ enhancers, kappa and mu intronic enhancers, and 3′ enhancers (see generally Paul W. E. (ed), Fundamental Immunology, 3rd Edition, Raven Press, New York (1993), pages 353-363; and U.S. Pat. No. 5,885,827).

The vector also can comprise a “selectable marker gene.” The term “selectable marker gene,” as used herein, refers to a nucleic acid sequence that allow cells expressing the nucleic acid sequence to be specifically selected for or against, in the presence of a corresponding selective agent. Suitable selectable marker genes are known in the art and described in, e.g., International Patent Application Publications WO 92/08796 and WO 94/28143; Wigler et al., Proc. Natl. Acad. Sci. USA, 77: 3567 (1980); O'Hare et al., Proc. Natl. Acad. Sci. USA, 78: 1527 (1981); Mulligan & Berg, Proc. Natl. Acad. Sci. USA, 78: 2072 (1981); Colberre-Garapin et al., J. Mol. Biol., 150:1 (1981); Santerre et al., Gene, 30: 147 (1984); Kent et al., Science, 237: 901-903 (1987); Wigler et al., Cell, 11: 223 (1977); Szybalska & Szybalski, Proc. Natl. Acad. Sci. USA, 48: 2026 (1962); Lowy et al., Cell, 22:817 (1980); and U.S. Pat. Nos. 5,122,464 and 5,770,359.

In certain embodiments, the nucleic acids encoding the polypeptides comprising the first and second components of an antigen-binding agent are provided to a population of cells in the form of an “episomal expression vector” or “episome,” which is able to replicate in a host cell, and persists as an extrachromosomal segment of DNA within the host cell in the presence of appropriate selective pressure (see, e.g., Conese et al., Gene Therapy 11: 1735-1742 (2004)). Representative commercially available episomal expression vectors include, but are not limited to, episomal plasmids that utilize Epstein Barr Nuclear Antigen 1 (EBNA1) and the Epstein Barr Virus (EBV) origin of replication (oriP). The vectors pREP4, pCEP4, pREP7, and pcDNA3.1 from Invitrogen (Carlsbad, Calif.), and pBK-CMV from Stratagene (La Jolla, Calif.) represent non-limiting examples of an episomal vector that uses T-antigen and the SV40 origin of replication in lieu of EBNA1 and oriP.

Other suitable vectors include integrating expression vectors, which may randomly integrate into the host cell's DNA, or may include a recombination site to enable the specific recombination between the expression vector and the host cells chromosome. Such integrating expression vectors may utilize the endogenous expression control sequences of the host cell's chromosomes to effect expression of the desired protein. Examples of vectors that integrate in a site specific manner include, for example, components of the flp-in system from Invitrogen (Carlsbad, Calif.) (e.g., pcDNA™5/FRT), or the cre-lox system, such as can be found in the pExchange-6 Core Vectors from Stratagene (La Jolla, Calif.). Examples of vectors that randomly integrate into host cell chromosomes include, for example, pcDNA3.1 (when introduced in the absence of T-antigen) from Invitrogen (Carlsbad, Calif.), and pCI or pFN10A (ACT) Flexi® from Promega (Madison, Wis.).

Viral vectors also can be used. Representative commercially available viral expression vectors include, but are not limited to, the adenovirus-based Per.C6 system available from Crucell, Inc. (Leiden, The Netherlands), the lentiviral-based pLP1 from Invitrogen (Carlsbad, Calif.), and the retroviral vectors pFB-ERV plus pCFB-EGSH from Stratagene (La Jolla, Calif.).

The first nucleic acid sequence and the set of second nucleic acid sequences can be provided to the population of cells on the same vector (i.e., in cis). This can be accomplished, for example, by cloning the first nucleic acid sequence into a vector library comprising a set of second nucleic acid sequences. A bidirectional promoter can be used to control expression of the first nucleic acid sequence and the set of second nucleic acid sequences. In another embodiment, a unidirectional promoter can control expression of the first nucleic acid sequence and the set of second nucleic acid sequences. In certain embodiments, the first nucleic acid sequence and the set of second nucleic acid sequences are separated by an internal ribosome entry site (IRES) to generate a bicistronic mRNA, which upon translation results in a set of antigen-binding agents comprising a first component and a second component that are not covalently linked. In other embodiments, the first nucleic sequence and a second nucleic acid sequence are translated as a single polypeptide, thereby resulting in a set of antigen-binding agents comprising a first component and a second component that are covalently linked.

The first nucleic acid sequence and the set of second nucleic acid sequences alternatively can be provided to the population of cells on separate vectors (i.e., in trans). The vector comprising the first nucleic acid sequence can comprise the same or different expression control sequences as the vector comprising the set of second nucleic acid sequences. The separate vectors can be provided to cells simultaneously or sequentially.

The vectors comprising the nucleic acids encoding polypeptides comprising a first component and a second component of an antigen-binding agent can be introduced into a host cell that is capable of expressing the polypeptides, including any suitable prokaryotic or eukaryotic cell. Preferred host cells are those that can be easily and reliably grown, have reasonably fast growth rates, have well characterized expression systems, and can be transformed or transfected easily and efficiently. In one embodiment of the invention, either the first nucleic acid sequence or a second nucleic acid sequence is endogenous to a cell, while the other nucleic acid sequence is introduced into the cell by transfection, transformation, or transduction. In other words, it is not required that both the first nucleic acid sequence and the set of second nucleic acid sequences are exogenous to the cell.

Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Erwinia. Particularly useful prokaryotic cells include the various strains of Escherichia coli (e.g., K12, HB101 (ATCC No. 33694), DH5α, DH10, MC1061 (ATCC No. 53338), and CC102).

Preferably, the vectors comprising the nucleic acids encoding polypeptides comprising a first component and a second component of an antigen-binding agent are introduced into a eukaryotic cell. Suitable eukaryotic cells are known in the art and include, for example, yeast cells, insect cells, and mammalian cells. Examples of suitable yeast cells include those from the genera Hansenula, Kluyveromyces, Pichia, Rhino-sporidium, Saccharomyces, and Schizosaccharomyces. Preferred yeast cells include, for example, Saccharomyces cerivisae and Pichia pastoris.

Suitable insect cells are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993). Preferred insect cells include Sf-9 and HI5 (Invitrogen, Carlsbad, Calif.).

Preferably, mammalian cells are utilized in the invention. In embodiments where mammalian cells are employed, somatic hypermutation may or may not be used in conjunction with the method of identifying an antigen-binding agent, i.e., the invention does not require SHM when performed using mammalian cells. In this regard, the invention also provides a method of identifying an antigen-binding agent that binds to an antigen of interest, which comprises providing a population of mammalian cells containing a first nucleic acid sequence and a set of second nucleic acid sequences, wherein the first nucleic acid sequence encodes a first polypeptide comprising a first component of an antigen-binding agent, wherein the set of second nucleic acid sequences comprises second nucleic acid sequences encoding second polypeptides comprising second components of antigen-binding agents, and wherein (1) the first component and (2) a second component together form an antigen-binding agent, (b) maintaining the population of mammalian cells under conditions wherein the first nucleic acid sequence and the set of second nucleic acid sequences are expressed to produce a set of antigen-binding agents, and (c) identifying an antigen-binding agent that binds to the antigen of interest.

A number of suitable mammalian host cells are known in the art and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, mouse L-929 cells, and BHK or HaK hamster cell lines, all of which are available from the ATCC. The selection of suitable mammalian host cells and methods for transformation, culture, amplification, screening, and purification of cells are known in the art.

In a preferred embodiment, the mammalian cell is a human cell. For example, the mammalian cell can be a human lymphoid or lymphoid derived cell line, such as a cell line of pre-B lymphocyte origin. Examples of human lymphoid cells lines include without limitation RAMOS(CRL-1596), Daudi (CCL-213), EB-3 (CCL-85), DT40 (CRL-2111), 18-81 (Jack et al., Proc. Natl. Acad. Sci. USA, 85: 1581-1585 (1988)), Raji cells (CCL-86), and derivatives thereof.

The nucleic acid sequence encoding a polypeptide comprising a first component or a second component of an antigen-binding agent may be introduced into a cell by “transfection,” “transformation,” or “transduction.” “Transfection,” “transformation,” or “transduction” as used herein, refers to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al. Mol. Cell. Biol., 7: 2031-2034 (1987)). Phage or viral vectors can be introduced into host cells, after growth of infectious particles in packaging cells that are commercially available.

In certain embodiments, the first and/or second components of the antigen-binding agent are chimeric molecules coupled in frame to a suitable transmembrane domain in order to provide cell-surface display of the antigen-binding agent (e.g., an antibody). For example, for expression in eukaryotic cells, a MHC type 1 transmembrane domain, such as that from H2kk (including peri-transmembrane domain, transmembrane domain, and cytoplasmic domain; NCBI Gene Accession number AK153419), can be coupled in frame to the first and/or second components of the antigen-binding agent using standard molecular biology techniques. Likewise, the surface expression of proteins in prokaryotic cells (such as E. coli and Staphylococcus), insect cells, and yeast is well established in the art. For reviews, see, for example, Winter et al., Annu. Rev. Immunol., 12: 433-55 (1994); Pluckthun, A., Bio/Technology, 9: 545-551 (1991); Gunneriusson et al., J. Bacteriol., 78: 1341-1346 (1996); Ghiasi et al., Virology, 185: 187-194 (1991); Boder and Wittrup, Nat. Biotechnol., 15: 553-557 (1997); and Mazor et al., Nat. Biotech., 25(5): 563-565 (2007).

In other embodiments, a surface displayed antigen-binding agent can be created through the secretion and then binding (or association) of secreted components on the cell surface. Conjugation of the antigen-binding agent to the cell membrane can occur either during protein synthesis or after one or more components of the antigen-binding agent have been secreted from the cell. Conjugation can occur via covalent linkage, by binding interactions (e.g., mediated by specific binding members) or a combination of covalent and non-covalent linkage.

In accordance with the invention, the antigen-binding agent that binds to an antigen of interest may be identified by any suitable method. In one aspect, the method comprises the steps of (i) providing the antigen of interest to the population of cells under conditions whereby the antigen of interest can bind to the antigen-binding agents, and (ii) identifying a sub-population of cells comprising the first nucleic acid sequence and the set of second nucleic acid sequences that are expressed to produce antigen-binding agents that bind the antigen of interest.

In certain embodiments, the invention provides a method of enriching the sub-population by (iii) separating the sub-population of cells comprising the first nucleic acid sequence and the set of second nucleic acid sequences that are expressed to produce antigen-binding agents that bind the antigen of interest with a desired affinity from cells that comprise the first nucleic acid sequence and the set of second nucleic acid sequences that are expressed to produce antigen-binding agents that either (1) do not bind the antigen of interest or (2) do not bind the antigen of interest with a desired affinity.

In other embodiments, the method of enriching the sub-population of cells comprises (iii) separating the sub-population of cells comprising the first nucleic acid sequence and the set of second nucleic acid sequences that are expressed to produce antigen-binding agents that bind an epitope of the antigen of interest with a desired affinity from cells that comprise the first nucleic acid sequence and the set of second nucleic acid sequences that are expressed to produce antigen-binding agents that either (1) do not bind the epitope of the antigen of interest or (2) do not bind the epitope of the antigen of interest with a desired affinity.

In still other embodiments, the method of enriching the sub-population of cells comprises (iii) separating the sub-population of cells comprising the first nucleic acid sequence and the set of second nucleic acid sequences that are expressed to produce antigen-binding agents that bind the antigen of interest from cells that comprise the first nucleic acid sequence and the set of second nucleic acid sequences that are expressed to produce antigen-binding agents that either (1) do not produce antigen-binding agents at a desired expression level, (2) do not produce antigen-binding agents having a desired stability, (3) do not produce antigen-binding agents having a desired functional activity, or (4) do not produce antigen-binding agents having a desired catalytic activity.

The method of enriching the sub-population of cells can alternatively comprise separating the sub-population of cells comprising the first nucleic acid sequence and the set of second nucleic acid sequences that are expressed to produce antigen-binding agents that bind the antigen of interest and which cross-react with a second antigen of interest with a desired affinity from cells that comprise the first nucleic acid sequence and the set of second nucleic acid sequences that are expressed to produce antigen-binding agents that either (1) do not cross-react with the second antigen of interest or (2) do not cross-react with the second antigen of interest with a desired affinity. Such embodiments of the invention may be useful, for example, when it is desired that the antigen-binding agent binds to more than one antigen of interest.

Any art-recognized assay can be utilized to identify a sub-population of cells which expresses an antigen-binding agent that binds the antigen of interest with a desired affinity or which binds a particular epitope of the antigen. Such methods include, for example, fluorescence activated cell sorting (FACS), separable beads (e.g., magnetic beads), antigen panning, and/or ELISA (see, e.g., Janeway et al. (eds.), Immunobiology, 5^{th} ed., Garland Publishing, New York, N.Y., 2001). Preferably, FACS is used to identify a sub-population of cells which expresses an antigen-binding agent of interest.

FACS staining protocols and screening methodologies, including high-throughput systems, are well known in the art. Exemplary screening assays that can be used in the context of the inventive method are described in, for example, U.S. Patent Application Publication 2009/0075378 A1 and International Patent Application Publications WO 08/103,475 and WO 08/103,474.

In one embodiment, FACS can be used to screen a library of cells expressing surface displayed antigen-binding agents that are undergoing, or have undergone, SHM mediated diversity. In this approach, a cell surface displayed library is used, and the displayed antigen-binding agents are first incubated with an antigen in solution. Typically, the antigen of interest is labeled (e.g., fluorescently or biotinylated). The FACS instrument is able to separate the high affinity antigen-binding members of the library, which have greater fluorescence intensity, from the lower affinity members. The use of optimized binding protocols in conjunction with FACS-based selection has been shown to be capable of evolving antibodies with up to femtomolar affinities (see, e.g., Boder et al., Proc. Natl. Acad. Sci. USA, 97: 10701-10705 (2000); Boder et al., Meth. Enzymol., 328: 430-444 (2000); and VanAntwerp et al., Biotechnol. Prog., 16: 31-37 (2000)).

In order to effectively select and rapidly evolve antigen-binding agents which have high affinity to an antigen of interest, protocols can be established that can facilitate the isolation of antigen-binding agents with a broad range of affinities to the antigens of interest, and yet eliminate those that bind to labeling or coupling reagents. These protocols involve both a progression in the stringency of the cell population selected, and a decrease in the concentration and density of the antigen presented to the cells.

With respect to the stringency or fraction of the total cell population collected during each round of selection, initial screens will generally use relatively low discrimination factors in order to capture as many antigen-binding agents as possible that possess small incremental improvements in binding characteristics. For example, a typical initial sort may capture the top 10%, top 5%, or top 2% of all cells that bind an antigen of interest. Large improvements in affinity may be the result of combinations of mutations, each of which contribute small additive effects to overall affinity (Hawkins et al., J. Mol. Biol., 234: 958-964 (1993)). Therefore, recovery of all library clones with even marginally improved affinities (2-3 fold) is desirable during the early stages of library screening, and sorting gates can be optimized to recover as many clones as possible with minimum sacrifice in enrichment.

Following the first round of sorting, the collected cells can be re-grown to amplify the population and then resorted. At this, and subsequent stages of sorting, greater enrichments are possible since more copies of each desirable clone are present within the examined cell population. For example only about the top 1%, top 0.5%, top 0.2%, or top 0.1% of the cells in the population may be selected in order to identify significantly improved clones. With respect to establishing optimal binding and selection strategies, first generation hits typically have low affinities and relatively rapid “off” rates. For example, Sagawa et al., Mol. Immunol., 39: 801-808 (2003), observed that the apparent affinity for germline antibodies is typically in the range of 2×10⁴ to 5×10⁶ M⁻¹, but that this affinity increases to around 10⁹ M⁻¹ during affinity maturation due to an effect mediated primarily by a decreased off rate (K_{off}).

After any round of selection, the cells may be expanded in culture for cell banking purposes or in order to determine the nucleic acid and/or amino acid sequences encoding one or more components of an identified antigen-binding agent. In this regard, the invention optionally involves sequencing the first nucleic acid sequence, one or more of the second nucleic acid sequences, one or both of the nucleic acid sequences encoding an identified antigen-binding agent, and/or one or more of the mutant nucleic acid sequences.

The nucleic acid sequences can be rescued and sequenced by any method known in the art. For example, total mRNA, or extrachromosomal plasmid DNA can be amplified by co-expression of SV40 T antigen (Heinzel et al., J. Virol., 62(10): 3738-3746 (1988)) and/or can be extracted from cells and used as a template for polymerase chain reaction (PCR) or reverse transcriptase (RT)-PCR to clone the nucleic acid sequence using appropriate primers. Nucleic acid sequences can be sub-cloned into a vector and expressed in E. coli. A tag (e.g., 6×-His tag) can be added to the carboxy terminus to facilitate protein purification using chromatography. The resulting data can be used to populate a database linking specific amino acid sequences with changes in one or more of the desired properties of an antigen-binding agent of interest. Such databases can then be used to recombine favorable mutations or to design a next generation polynucleotide library with targeted diversity in a newly identified region of interest.

When the antigen-binding agent is an antibody or fragment thereof, DNA can be extracted from a cell by PCR using variable heavy chain (V_{H}) leader region and/or variable light chain (V_{L}) leader region-specific sense primers and isotype-specific antisense primers. Alternatively, total RNA from selected sorted cell populations can be isolated and subjected to RT-PCR using variable heavy chain (V_{H}) leader region and/or variable light chain (V_{L}) leader region-specific sense primers and isotype-specific antisense primers. Clones can be sequenced using standard methodologies as described herein.

An antigen-binding agent provided by the inventive method can be screened for a desired property (e.g., a selectable or improved phenotype) using a variety of standard physiological, pharmacological, and biochemical procedures. Such assays include for example, biochemical assays such as binding assays, fluorescence polarization assays, solubility assays, folding assays, thermostability assays, proteolytic stability assays, and enzyme activity assays (see generally Glickman et al., J. Biomolecular Screening, 7(1): 3-10 (2002); Salazar et al., Methods. Mol. Biol., 230: 85-97 (2003)), as well as a range of cell based assays including signal transduction, motility, whole cell binding, flow cytometry, and fluorescent activated cell sorting (FACS) based assays. When the antigen-binding agent is an antibody or a fragment thereof, the phenotype/function of the antibody or fragment thereof can be further analyzed using any suitable technique, such as art-recognized assays (e.g., enzyme-linked immunosorbant assays (ELISA), enzyme-linked immunosorbant spot (ELISPOT assay), gel detection and fluorescent detection of mutated IgH chains, Scatchard analysis, Biacore analysis, western blots, polyacrylamide gel (PAGE) analysis, radioimmunoassays, etc.), which can determine binding affinity, binding avidity, and other properties.

Once a desired antigen-binding agent is identified which binds to an antigen of interest, the antigen-binding portion may be used in the generation of a recombinant antibody or a fragment thereof (e.g., Fab, Fv, etc.) using either protein chemistry or recombinant DNA technology. Furthermore, although the two domains of the Fv fragment, V_{L} and V_{H}, are coded for by separate genes, the two domains can be joined, using recombinant methods, by a synthetic linker that enables them to be made as a single protein chain in which the V_{L} and V_{H} regions pair to form monovalent molecules (known as single chain Fv (scFv); see, e.g., Bird et al., Science, 242: 423-426 (1988); and Huston et al., Proc. Natl. Acad. Sci. USA, 85: 5879-5883 (1988); and Osbourn et al., Nat. Biotechnol., 16: 778 (1998)). Other forms of single chain antibodies, such as diabodies, are also encompassed by the invention. An antigen-binding fragment provided by the methods disclosed herein also can be linked to human immunoglobulin constant region cDNA or genomic sequences, in order to generate expression vectors encoding complete IgG molecules or other isotypes.

One of ordinary skill in the art will appreciate that, once identified, the antigen-binding agent can be inserted into other molecules (e.g., polypeptides) to generate novel molecules which bind the antigen of interest. In this regard, the invention comprises a method of producing a polypeptide which binds an antigen of interest, which comprises inserting the antigen-binding agent into a polypeptide. Such novel antigen-binding molecules can be generated using routine molecular biology techniques known in the art. For example, the antigen-binding agent, the amino acid sequence of the antigen-binding agent, one or both of the nucleic acid sequences encoding the antigen-binding agent (or antigen-binding fragments of any of the foregoing) can be inserted into a different molecule (e.g., a polypeptide or a polynucleotide) to generate a recombinant molecule that binds to the antigen of interest. In a preferred embodiment, a CDR3 or a variable region (HC or LC) identified by the methods described herein can transplanted (grafted) into another molecule, such as an antibody or non-antibody polypeptide, using either protein chemistry or recombinant DNA technology. In another embodiment, the entire variable region of a LC and/or a HC identified by the methods described herein can be transplanted in place of the variable region of a LC and/or a HC of another antibody. In other embodiments, a transplanted molecule can comprise a single amino acid change that is present in the identified antigen-binding agent but is absent in the non-transplanted molecule, which change produces a desired property in the transplanted molecule. The aforementioned methods may be useful, for example, to generate an antibody comprising an Fc region of a different isotype or an Fc region that is conjugated to a protein or non-protein moiety (e.g., a fluorescent tag or a chemotherapeutic agent).

The invention also encompasses an antigen-binding agent identified by the methods described herein, as well as compositions comprising (i) an antigen-binding agent identified by the methods described herein and (ii) a carrier therefor. Preferably, the composition is a pharmaceutically acceptable (e.g., physiologically acceptable) composition, which comprises a carrier, preferably a pharmaceutically (e.g., physiologically acceptable) carrier and an antigen-binding agent. Any suitable carrier can be used within the context of the invention, and such carriers are well known in the art. The choice of carrier will be determined, in part, by the particular site to which the composition may be administered and the particular method used to administer the composition. The composition optionally can be sterile.

The invention further provides compositions comprising an antigen-binding agent and a carrier therefor, wherein the antigen-binding agent is provided by a method comprising: (a) providing a population of cells containing a first nucleic acid sequence and a set of second nucleic acid sequences, wherein the first nucleic acid sequence encodes a first polypeptide comprising a first component of an antigen-binding agent, which first nucleic acid sequence optionally has been prepared by subjecting a nucleic acid sequence to somatic hypermutation, wherein the set of second nucleic acid sequences comprises second nucleic acid sequences encoding second polypeptides comprising second components of antigen-binding agents, which second nucleic acid sequences optionally have been prepared by subjecting a library of nucleic acid sequences to somatic hypermutation, and wherein (1) the first component and (2) a second component together form an antigen-binding agent, and wherein the population of cells optionally expresses activation-induced cytidine deaminase (AID), (b) maintaining the population of cells under conditions wherein the first nucleic acid sequence and the set of second nucleic acid sequences are expressed to produce a set of antigen-binding agents, (c) identifying an antigen-binding agent that binds to the antigen of interest, and optionally, (d) subjecting one or both of the nucleic acid sequences encoding the identified antigen-binding agent to somatic hypermutation to provide mutant nucleic acid sequences encoding a desired antigen-binding agent that binds to the antigen of interest, with the proviso that at least one of the first nucleic acid sequence, the second nucleic acid sequences, and/or the nucleic acid sequences encoding the identified antigen-binding agent is subjected to somatic hypermutation.

The invention also encompasses utilizing the desired antigen-binding agent or a fragment thereof to produce additional copies of the antigen-binding agent or fragment thereof. In one embodiment, the invention comprises obtaining the amino acid sequence of the desired antigen-binding agent or fragment thereof and utilizing the obtained amino acid sequence to produce additional copies of the antigen-binding agent or fragment thereof. In another embodiment, the invention comprises utilizing the mutant nucleic acid sequences to produce additional copies of the desired antigen-binding agent or fragment thereof. In this respect, the additional copies of the desired antigen-binding agent or fragment thereof are produced by amplifying the mutant nucleic acid sequences to provide multiple copies of the mutant nucleic acid sequences, and expressing the multiple copies of the mutant nucleic acid sequences in a cell to produce the additional copies of the desired antigen-binding agent or fragment thereof. In another embodiment, the invention comprises obtaining the sequences of the mutant nucleic acid sequences and utilizing the obtained sequences to produce additional copies of the desired antigen-binding agent or fragment thereof. Utilizing the obtained sequences to prepare additional copies of the desired antigen-binding agent or fragment thereof desirably comprises preparing one or more nucleic acids having the obtained sequences and expressing the one or more nucleic acids in one or more cells to produce the additional copies of the desired antigen-binding agent or fragment thereof. Methods to obtain, amplify, sequence, and/or express a nucleic acid sequence encoding an antigen-binding agent or fragment thereof as set forth above in connection with other embodiments of the invention also are applicable to those same aspects of the aforesaid methods to produce additional copies of a desired antigen-binding agent or fragment thereof.

The following examples further illustrate the invention but should not be construed as in any way limiting its scope.

### Example 1

This example demonstrates a method for identifying cells which express an antigen-binding agent that binds to IL-17a in accordance with the inventive method.

A nucleic acid sequence encoding the heavy chain (HC) of a humanized reference IL-17 antibody, was cloned into an expression vector as described in, for example, U.S. Patent Application Publications 2009/0093024 A1 and 2009/0075378 A1. The expression vectors utilized in this example comprise pJ2 or pJ15 from DNA2.0 (Menlo Park, Calif.) as the vector backbone, and are described in more detail in U.S. Patent Application Publication 2009/0075378 A1. Briefly, the expression vectors contained the following elements operably linked together: (1) CMV promoter; (2) multicloning sites; (3) gene(s) of interest (e.g., a HC encoding gene); (4) terminator sequences, (3′ untranslated region, small intron and polyA signals from SV40 (“IVS pA”)); (5) Epstein Barr Virus (EBV) origin of replication (oriP) (preceded by optional intergenic spacer region); (6) SV40 immediate early promoter (pSV), eukaryotic selectable marker such as blasticidin S deaminase (bsd), hygromycin phosphotransferase (hyg) or puromycin-N-acetyl-transferase, and IVS pA; (7) prokaryotic origin of replication (ColE1 ori); (8) prokaryotic selectable marker such as beta lactamase (bla) gene or kanamycin (kan); (9) gene fragment for copy number determination (such as beta actin or glucose-6-phosphate dehydrogenase (G6PDH); and (10) Ig enhancers.

The vector comprising the HC of a reference IL-17 antibody was subjected to SHM as described in, for example, U.S. Patent Application Publications 2009/0093024 A1 and 2009/0075378 A1 to generate the “mature HC,” or “HCₘₐₜᵤᵣₑ”. A library of nucleic acids, each of which encode a kappa (K) light chain (LC), was prepared as described in, for example, U.S. Patent Application Publications 2009/0093024 A1 and 2009/0075378 A1. HEK293 cells were co-transfected with the vectors encoding the mature HC and the library of kappa LCs, and subjected to stable selection. AID was pulsed transiently in the HEK293 cells before, during, and after the chain shuffling process.

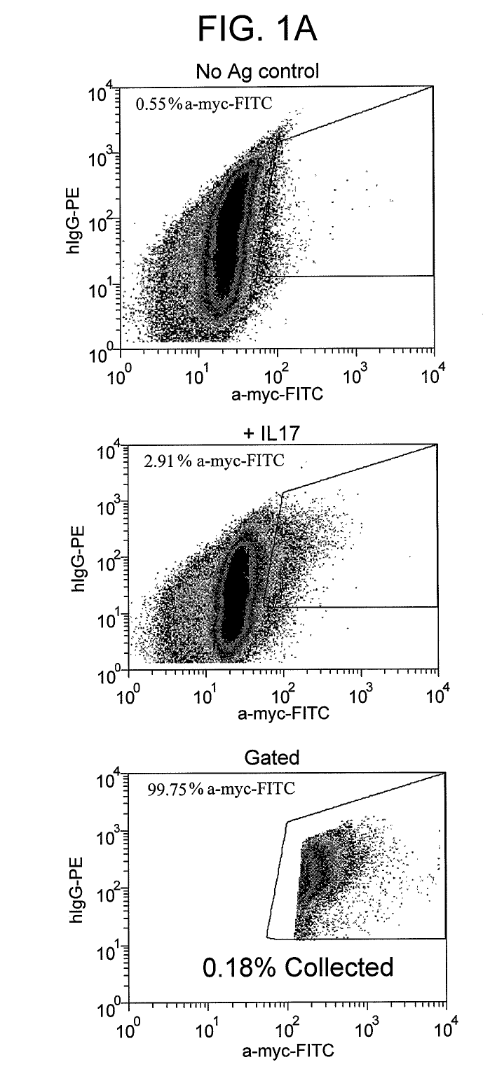

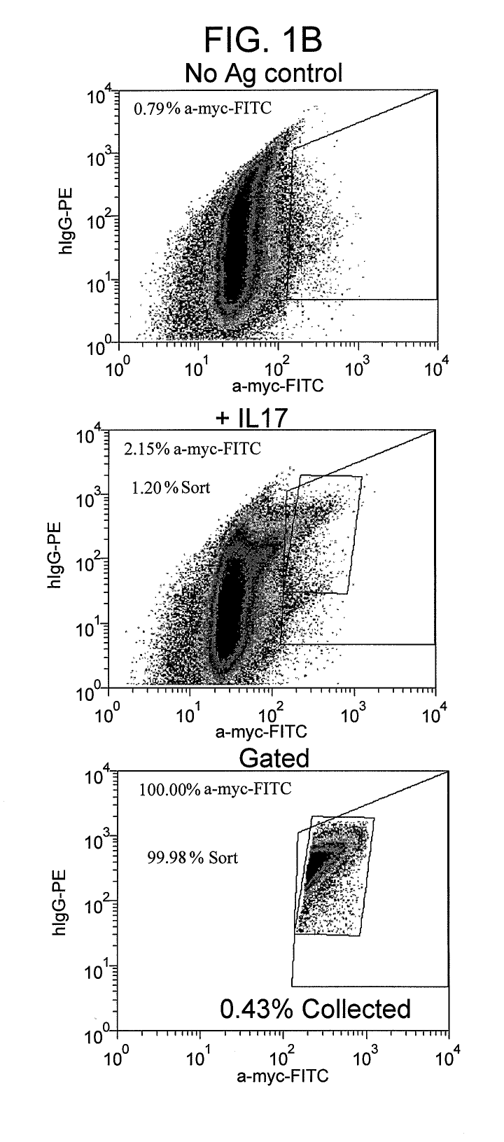

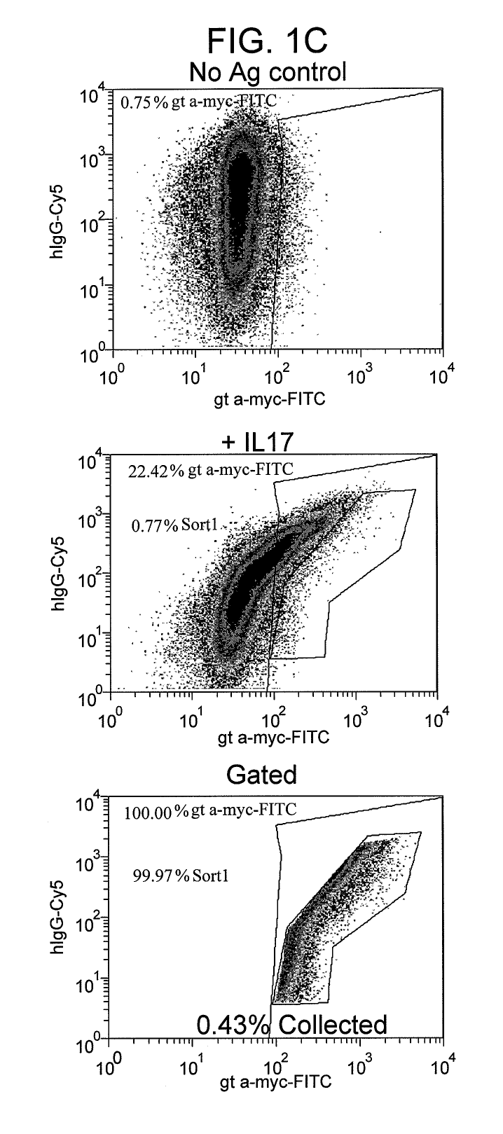

In the first round of screening to identify HCₘₐₜᵤᵣₑ/LC combinations which reconstitute high affinity binding to IL-17a, the stably transfected cells were split into two groups: (1) a control group which was left untreated and (2) a group which was incubated with 25 nM of myc-tagged IL-17a (IL-17a-myc). Both groups were sorted by FACS in two-dimensions using fluorescently labeled anti-IgG and anti-myc antibodies. 0.18% of the sorted cells were collected in round 1 as expressing HCₘₐₜᵤᵣₑ/LC combinations which bind to IL-17a (FIG. 1A).

For the second round of screening, the population of cells collected in round 1 was expanded, and a portion of the population was split into two treatment groups: (1) a control group which was left untreated and (2) a group which was incubated with 10 nM of IL-17a-myc. Both groups were sorted by FACS as described above. 0.43% of the sorted cells were collected in round 2 as expressing HCₘₐₜᵤᵣₑ/LC combinations which bind IL-17a (FIG. 1B).

The same procedure was followed in round three of screening, except that a portion of the population of cells collected in round 2 was left untreated and another portion was incubated with 3 nM of IL-17a prior to FACS sorting as described above. 0.43% of the sorted cells were collected in round 3 as expressing HCₘₐₜᵤᵣₑ/LC combinations which bind to IL-17a (FIG. 1C).

This example demonstrates a method of identifying a population of cells which express an antigen-binding agent encoded by a first nucleic acid sequence and a set of second nucleic acid sequences in accordance with the invention.

### Example 2

This example describes a method of determining the nucleic acid sequences encoding second polypeptides comprising second component light chains, which together with a first component HCₘₐₜᵤᵣₑ, form antigen-binding agents that bind to IL-17a with high affinity.

DNA was harvested from a portion of the positively gated cell population collected in round 1 and round 2 of the screen described in Example 1 by conventional methods (such as those described herein). Open reading frames of LCs were obtained by PCR, and the DNA sequences from individually cloned templates were obtained by conventional methods (such as those described herein). Alternatively, the episomal DNA harvested from a portion of the positively gated cell population was transformed into E. Coli, and DNA sequences from individual clones were obtained. A total of 187 DNA sequences were obtained from a portion of the positively gated cell population collected in the screen described in Example 1. The corresponding amino acid sequences of the obtained DNA sequences are provided as SEQ ID NOs: 15-201.

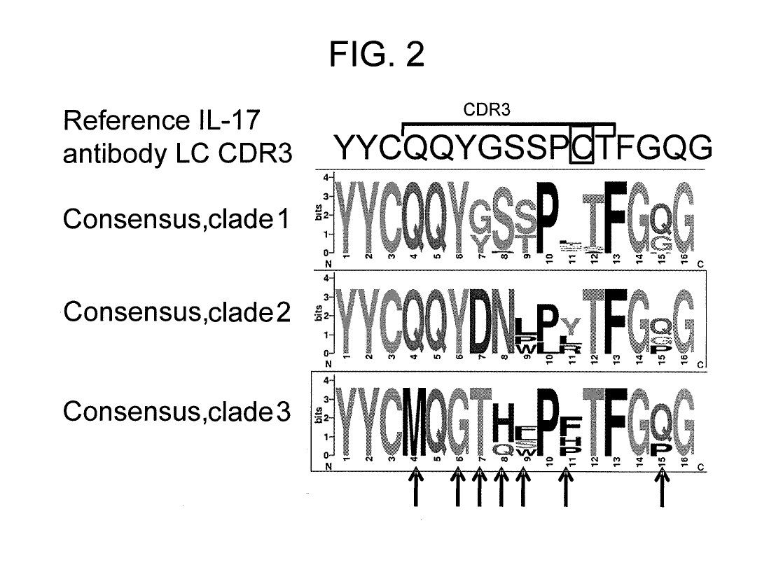

A comparison of the sequences from a portion of the positively gated cell population collected in round 1 of the screen described in Example 1 identified three clades of sequences. Certain amino acid residues in the CDR3 region of the LC were found to be well conserved not only among all three clades, but also in comparison to the CDR3 region of the reference IL-17 antibody LC sequence, in particular glutamine (Q) at position 5 and proline (P) at position 10 (FIG. 2).

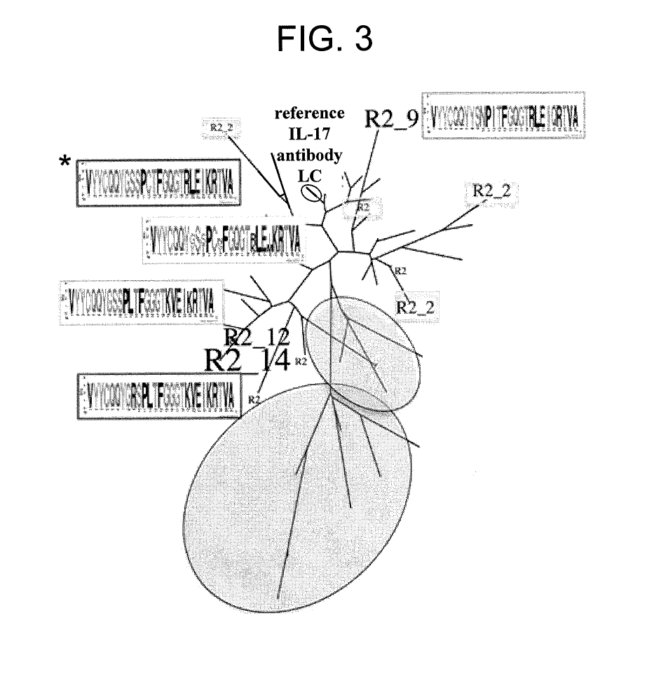

A comparison of the sequences from a portion of the positively gated cell population collected in round 2 of the screen described in Example 1 demonstrated that clade 2 and clade 3 identified in round 1 were no longer represented in the population, and that the diversity of clade 1 was expanded further (FIG. 3). Among the sequences of the diversified clade 1, two sub-clades made up greater than 50% of the acquired sequences, which sub-clades differed by one amino acid from one another and differed by four or five amino acids from the reference IL-17 antibody LC sequence (FIG. 3).

This example demonstrates a method for identifying the nucleotide sequences encoding second polypeptides comprising second component LCs of antigen-binding agents which bind to IL-17a with high affinity.

### Example 3

This example demonstrates a method for identifying cells which express antigen-binding agents that bind to IL-17a in accordance with the inventive method.

The nucleic acid sequence encoding the mature HC which subjected to SHM described in Example 1 was determined by standard methods (such as those described herein). The nucleic acid sequence of the germline sequence from which the mature HC was derived was also determined. A vector was then constructed comprising a “devolved” version of the mature HC wherein all or part of the variable region, except the CDR3 region, was devoid of SHM. This weaker version of the mature HC, termed “germline HC” or “HC_{germline},” was then co-transfected into HEK293 cells along with the library of nucleic acids encoding kappa LCs described in Example 1. AID was pulsed transiently in the HEK293 cells before, during, and after the chain shuffling process.

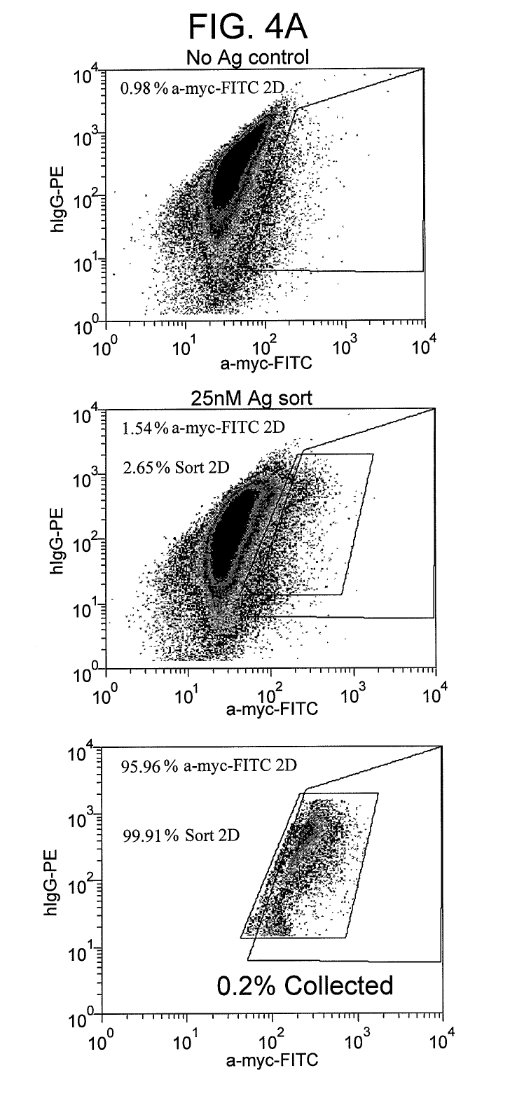

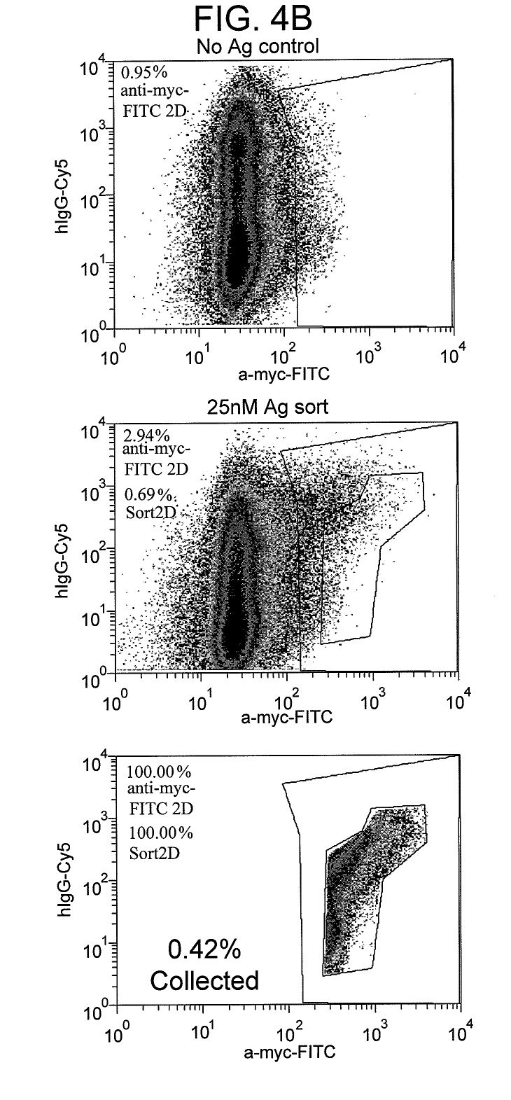

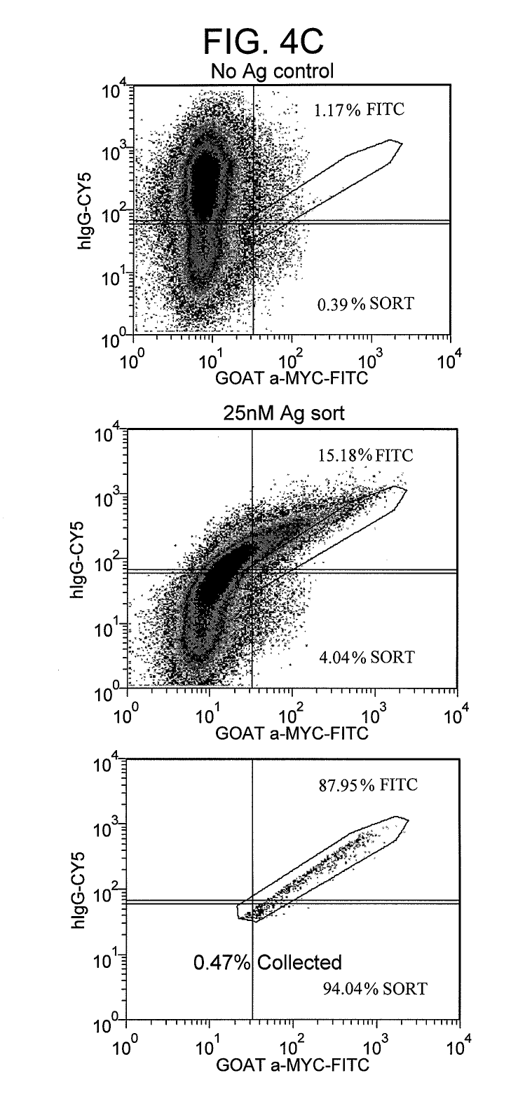

In the first round of screening to identify HC_{germline}/LC combinations which together form an antigen-binding agent that binds to IL-17a with high affinity, the stably transfected cells were split into two groups: (1) a control group which was left untreated, and (2) a group which was incubated with 25 nM of IL-17a-myc. Both groups were sorted by FACS in two-dimensions using fluorescently labeled anti-IgG and anti-myc antibodies. 0.2% of the sorted cells were collected in round 1 as expressing HC_{germline}/LC combinations which bind to IL-17a (FIG. 4A).

For the second round of screening, the population of cells collected in round 1 was expanded, and a portion of the population was split into two treatment groups: (1) a control group which was left untreated and (2) a group which was incubated with 25 nM of IL-17a-myc. Both groups were sorted by FACS as described above. 0.42% of the sorted cells were collected in round 2 as expressing HC_{germline}/LC combinations which bind to IL-17a (FIG. 4B).

A similar procedure was followed in round three of screening, wherein a portion of the population of cells collected in round 2 was left untreated and another portion was incubated with 25 nM of IL-17a prior to FACS sorting as described above. 0.47% of the sorted cells were collected in round 3 as expressing HC_{germline}/LC combinations which bind to IL-17a (FIG. 4C).

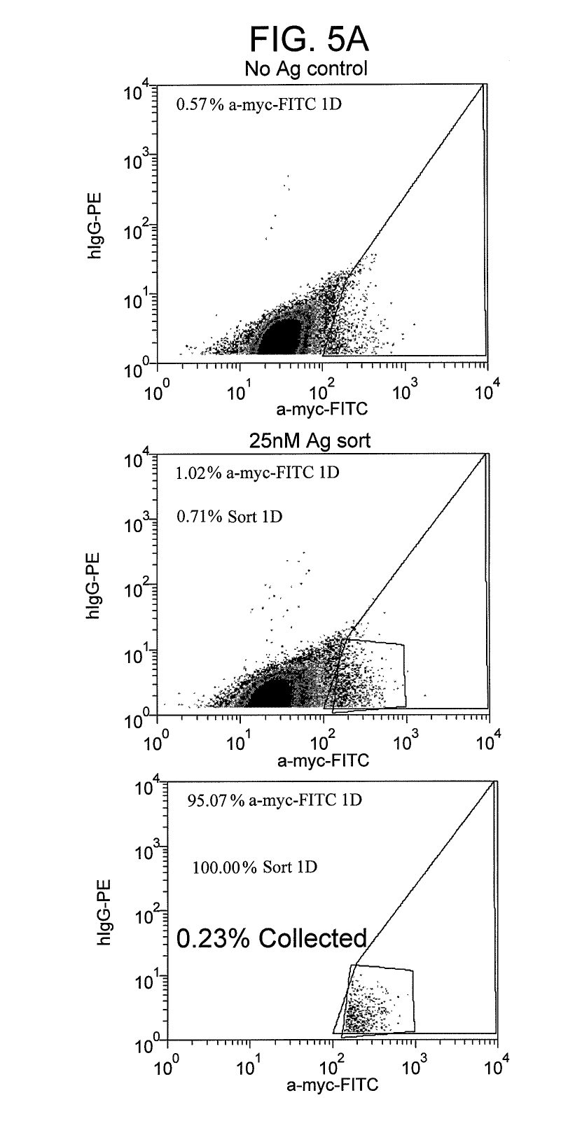

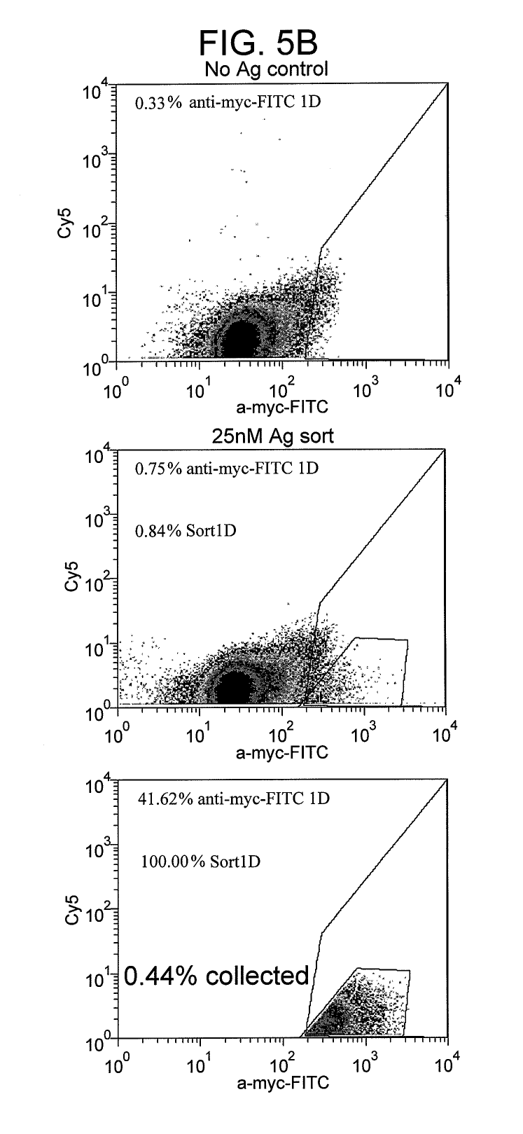

A separate set of experiments was performed similarly, except that the cells were sorted in only one-dimension using anti-myc antibodies (i.e., the anti-IgG sorting was eliminated). 0.23% of the sorted cells were collected in round 1 as expressing HC_{germline}/LC combinations which bind to IL-17a (FIG. 5A). In the second round of screening, 0.44% of the sorted cells were collected as expressing HC_{germline}/LC combinations which bind to IL-17a (FIG. 5B).

This example demonstrates a method of identifying a population of cells which express an antigen-binding agent encoded by a first nucleic acid sequence and a set of second nucleic acid sequences in accordance with the invention.

### Example 4

This example demonstrates that the LC sequences obtained in Example 3 using the germline HC as the first component of an antigen-binding agent were within the same three clades identified when mature HC was used.

The DNA sequences of the LCs identified in the screen described in Example 3 were determined as described in Example 2. A total of 97 DNA sequences were obtained from a portion of the positively gated cell population collected after sorting in two-dimensions in the screen described in Example 3. The corresponding amino acid sequences of the obtained DNA sequences are provided as SEQ ID NOs: 202-298. A total of 59 DNA sequences were obtained from a portion of the positively gated cell population collected after sorting in one-dimension in the screen described in Example 3. The corresponding amino acid sequences of the obtained DNA sequences are provided as SEQ ID NOs: 299-357.

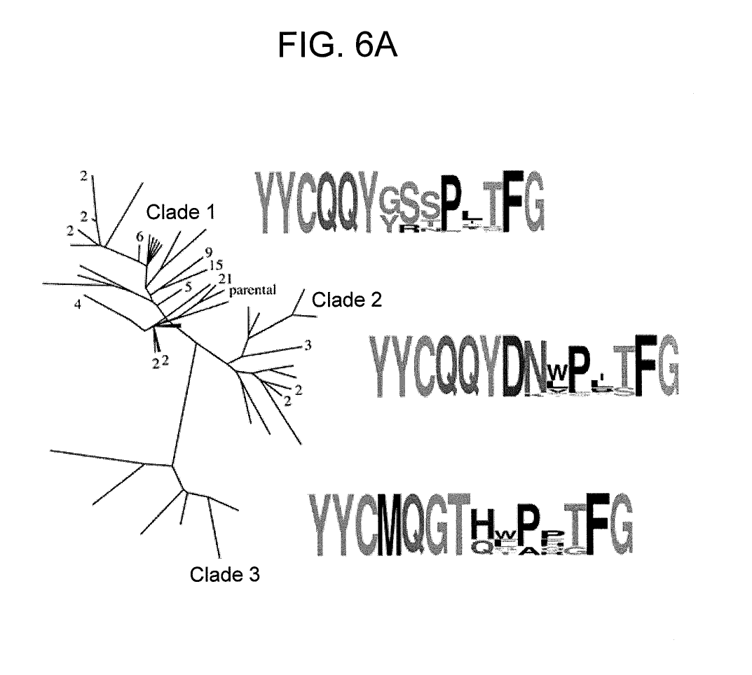

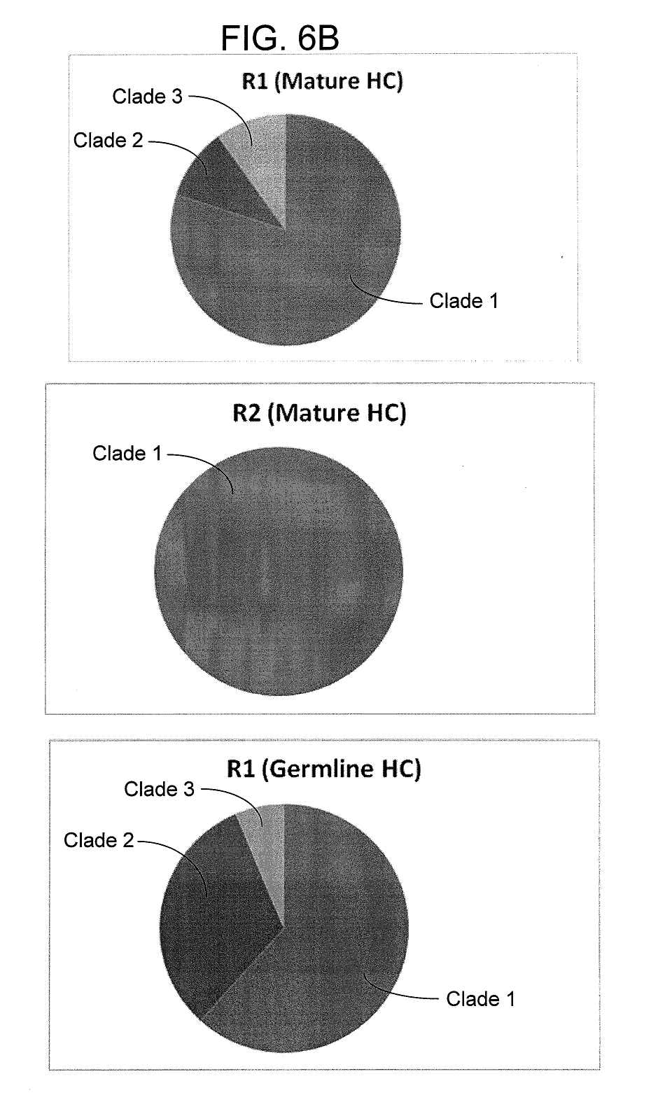

A comparison of the LC sequences obtained from a portion of the positively gated cell population collected after round 1 of sorting in two-dimensions in the screen described in Example 3 indicated that the LC sequences obtained using HC_{germline} as the first component of an antigen-binding agent fell within the same three clades identified when HCₘₐₜᵤᵣₑ was used (FIG. 6A). The obtained nucleic acid sequences were not identical among the HC_{germline} and HCₘₐₜᵤᵣₑ strategies, but were generally found to be homologous in sequence (FIG. 6B).

This example demonstrates the nucleic acid sequences encoding second component LCs of an antigen-binding agent are similar whether a mature HC or a germline HC is used as the first component of an antigen-binding agent.

### Example 5

This example demonstrates a method for identifying antigen-binding agents that bind to IL-17a in accordance with the inventive method.

The nucleic acid sequence encoding the LC of a reference IL-17 antibody was cloned into an expression vector as described in Example 1 and subjected to SHM to generate a “mature LC,” or “LCₘₐₜᵤᵣₑ”. The nucleic acid sequence encoding the mature LC was determined by standard methods (such as those described herein). The nucleic acid sequence of the germline sequence from which the mature LC was derived was also determined. A vector then was constructed comprising a “devolved” version of the mature LC wherein all of the variable region except for the CDR3 region was devoid of SHM. This weaker version of a mature LC was termed “germline LC” or “LC_{germline}.” A library of nucleic acids, each of which encode a HC, was prepared as described in, for example, U.S. Patent Application Publication 2009/0093024 A1.

HEK293 cells were stably co-transfected with the vectors encoding the germline LC and the library of HCs. AID was pulsed transiently in the HEK293 cells before, during, and after the chain shuffling process.

The cells were incubated with IL-17a and sorted by FACS as described in Example 1 and Example 3 in order to identify novel HCs which can pair with the fixed LC_{germline} to reconstitute high affinity binding to antigen.

This example demonstrates that a first component LC can be used to identify novel second component HCs which together form antigen-binding agents which bind to IL-17a with high affinity.

### Example 6

This example demonstrates a method for identifying mammalian cells which express an antigen-binding agent that binds to IL-17a in accordance with the inventive method.

A nucleic acid sequence encoding the heavy chain (HC) of a humanized reference IL-17 antibody is cloned into an expression vector as described in Example 1. A library of nucleic acids, each of which encodes a kappa (κ) light chain (LC), is prepared as described in, for example, U.S. Patent Application Publications 2009/0093024 A1 and 2009/0075378 A1. HEK293 cells are co-transfected with the vectors encoding the HC and the library of kappa LCs, and are subjected to stable selection.

A screen to identify HC/LC combinations which reconstitute high affinity binding to IL-17a is performed by splitting the stably transfected cells into two groups: (1) a control group which is left untreated and (2) a group which is incubated with 25 nM of myc-tagged IL-17a (IL-17a-myc). Both groups are then sorted by FACS in two-dimensions using fluorescently labeled anti-IgG and anti-myc antibodies. A fraction of the sorted cells which express HC/LC combinations which bind to IL-17a are collected for further enrichment in a second round of screening.

For the second round of screening, the population of cells which express HC/LC combinations which bind to IL-17a is expanded and then split into two treatment groups: (1) a control group which is left untreated and (2) a group which is incubated with 10 nM of IL-17a-myc. Both groups are then sorted by FACS and a fraction of the sorted cells which express HC/LC combinations which bind to IL-17a are collected as described above. The collected cells can be used for further enrichment in a third round of screening, if desired. In addition, a portion of the cells can be cryopreserved, or a portion of the cells can be utilized to obtain the DNA sequences encoding HC/LC combinations as described in Example 2 and Example 4 herein.

This example demonstrates a method of identifying a population of mammalian cells which express an antigen-binding agent encoded by a first nucleic acid sequence and a set of second nucleic acid sequences in accordance with the invention.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of identifying an antigen-binding agent that binds to an antigen of interest, which method comprises:
(a) providing a population of cells containing a first nucleic acid sequence and a set of second nucleic acid sequences,
wherein the first nucleic acid sequence encodes a first polypeptide comprising a first component of an antigen-binding agent, which first nucleic acid sequence optionally has been prepared by subjecting a nucleic acid sequence to somatic hypermutation,
wherein the set of second nucleic acid sequences comprises second nucleic acid sequences encoding second polypeptides comprising second components of antigen-binding agents, which second nucleic acid sequences optionally have been prepared by subjecting a library of nucleic acid sequences to somatic hypermutation, and
wherein (1) the first component and (2) a second component together form an antigen-binding agent, and wherein the population of cells optionally expresses activation-induced cytidine deaminase (AID),
(b) maintaining the population of cells under conditions wherein the first nucleic acid sequence and the set of second nucleic acid sequences are expressed to produce a set of antigen-binding agents,
(c) identifying an antigen-binding agent that binds to the antigen of interest, and
(d) optionally subjecting one or both of the nucleic acid sequences encoding the identified antigen-binding agent to somatic hypermutation to provide mutant nucleic acid sequences encoding a desired antigen-binding agent that binds to the antigen of interest,
with the proviso that at least one of the first nucleic acid sequence, the second nucleic acid sequences, and/or the nucleic acid sequences encoding the identified antigen-binding agent is subjected to somatic hypermutation.

2. The method of claim 1, wherein
the first nucleic acid sequence has not been prepared by subjecting a nucleic acid sequence to somatic hypermutation, and
the second nucleic acid sequences have been prepared by subjecting a library of nucleic acid sequences to somatic hypermutation.

3. The method of claim 1, wherein
the first nucleic acid sequence has been prepared by subjecting a nucleic acid sequence to somatic hypermutation, and
the second nucleic acid sequences have not been prepared by subjecting a library of nucleic acid sequences to somatic hypermutation.

4. The method of claim 1, wherein
the first nucleic acid sequence has been prepared by subjecting a nucleic acid sequence to somatic hypermutation, and
the second nucleic acid sequences have been prepared by subjecting a library of nucleic acid sequences to somatic hypermutation.

5. The method of claim 1, wherein
the first nucleic acid sequence has not been prepared by subjecting a nucleic acid sequence to somatic hypermutation, and
the second nucleic acid sequences have not been prepared by subjecting a library of nucleic acid sequences to somatic hypermutation.

6. The method of claim 1, further comprising repeating steps (a), (b), (c), and optionally (d), except that, in the repeated steps, the first nucleic acid sequence comprises one of the mutant nucleic acid sequences encoding the desired antigen-binding agent provided by initial steps (a), (b), (c), and (d).

7. The method of claim 1, wherein identifying the antigen-binding agent comprises:
(i) providing the antigen of interest to the population of cells under conditions whereby the antigen of interest can bind to the antigen-binding agents, and
(ii) identifying a sub-population of cells comprising the first nucleic acid sequence and the set of second nucleic acid sequences that are expressed to produce antigen-binding agents that bind the antigen of interest.

8. The method of claim 7, which further comprises enriching the sub-population of cells by:
(iii) separating the sub-population of cells comprising the first nucleic acid sequence and the set of second nucleic acid sequences that are expressed to produce antigen-binding agents that bind the antigen of interest with a desired affinity from cells that comprise the first nucleic acid sequence and the set of second nucleic acid sequences that are expressed to produce antigen-binding agents that either (1) do not bind the antigen of interest or (2) do not bind the antigen of interest with a desired affinity.

9. The method of claim 7, which further comprises enriching the sub-population of cells by:
(iii) separating the sub-population of cells comprising the first nucleic acid sequence and the set of second nucleic acid sequences that are expressed to produce antigen-binding agents that bind an epitope of the antigen of interest with a desired affinity from cells that comprise the first nucleic acid sequence and the set of second nucleic acid sequences that are expressed to produce antigen-binding agents that either (1) do not bind the epitope of the antigen of interest or (2) do not bind the epitope of the antigen of interest with a desired affinity.

10. The method of claim 7, which further comprises enriching the sub-population of cells by:
(iii) separating the sub-population of cells comprising the first nucleic acid sequence and the set of second nucleic acid sequences that are expressed to produce antigen-binding agents that bind the antigen of interest and which cross-react with a second antigen of interest with a desired affinity from cells that comprise the first nucleic acid sequence and the set of second nucleic acid sequences that are expressed to produce antigen-binding agents that either (1) do not cross-react with the second antigen of interest or (2) do not cross-react with the second antigen of interest with a desired affinity.

11. The method of claim 7, which further comprises enriching the sub-population of cells by:
(iii) separating the sub-population of cells comprising the first nucleic acid sequence and the set of second nucleic acid sequences that are expressed to produce antigen-binding agents that bind the antigen of interest from cells that comprise the first nucleic acid sequence and the set of second nucleic acid sequences that are expressed to produce antigen-binding agents that either (1) do not produce antigen-binding agents at a desired expression level, (2) do not produce antigen-binding agents having a desired stability, (3) do not produce antigen-binding agents having a desired functional activity, or (4) do not produce antigen-binding agents having a desired catalytic activity.

12. The method of claim 7, wherein the sub-population of cells is identified using fluorescence activated cell sorting (FACS), separable beads, antigen panning, and/or ELISA.

13. The method of claim 1, further comprising sequencing the first nucleic acid sequence, one or more of the second nucleic acid sequences, one or both of the nucleic acid sequences encoding an identified antigen-binding agent, and/or one or more of the mutant nucleic acid sequences.

14. The method of claim 1, wherein the antigen-binding agent is an antibody, an antibody conjugate, or an antigen-binding fragment thereof.

15. The method of claim 1, wherein the first polypeptide comprising a first component of an antigen-binding agent is an antibody heavy chain or a fragment thereof, and each of the second polypeptides comprising second components of antigen-binding agents is an antibody light chain or fragment thereof.

16. The method of claim 1, wherein the first polypeptide comprising a first component of an antigen-binding agent is an antibody light chain or a fragment thereof, and each of the second polypeptides comprising second components of antigen-binding agents is an antibody heavy chain or fragment thereof.

17. The method of claim 15, wherein the antibody light chain is a kappa (κ) light chain.

18. The method of claim 15, wherein the antibody light chain is a lambda (λ) light chain.

19. The method of claim 1, wherein the first component of an antigen-binding agent is obtained from a human antibody, a non-human antibody, or a chimeric antibody.

20. The method of claim 1, wherein the second component of an antigen-binding agent is obtained from a human antibody, a non-human antibody, or a chimeric antibody.

21. The method of claim 1, wherein the first nucleic acid sequence and at least one of the second nucleic acid sequences are provided on the same nucleic acid molecule.

22. The method of claim 1, wherein the first nucleic acid sequence and the set of second nucleic acid sequences are provided on different nucleic acid molecules.

23. The method of claim 1, wherein the library is synthetically generated.

24. The method of claim 1, wherein the first nucleic acid sequence has been modified as compared to a corresponding wild-type nucleic acid sequence to increase or decrease the density of somatic hypermutation cold spots and/or somatic hypermutation hot spots so as to increase or decrease the susceptibility of the nucleic acid sequence to somatic hypermutation.

25. The method of claim 1, wherein one or more of the second nucleic acid sequences have been modified as compared to a corresponding wild-type nucleic acid sequence to increase or decrease the density of somatic hypermutation cold spots and/or somatic hypermutation hot spots so as to increase or decrease the susceptibility of the nucleic acid sequence to somatic hypermutation.

26. The method of claim 1, wherein the cells are eukaryotic cells.

27. The method of claim 1, wherein the first nucleic acid sequence and/or the set of second nucleic acid sequences are in the form of a vector.

28. The method of claim 27, wherein the vector is a replicable genetic display package.

29. The method of claim 28, wherein the replicable genetic display package is a viral vector or a bacteriophage.

30. The method of claim 27, wherein the first nucleic acid sequence and the set of second nucleic acid sequences are provided to the population of cells on the same vector.

31. The method of claim 27, wherein the first nucleic acid sequence and the set of second nucleic acid sequences are provided to the population of cells on separate vectors.

32. The method of claim 1, wherein the first nucleic acid sequence and the set of second nucleic acid sequences are provided to cells simultaneously or sequentially.

33. The method of claim 1, further comprising utilizing the desired antigen-binding agent or a fragment thereof to produce additional copies of the antigen-binding agent or fragment thereof.

34. The method of claim 1, further comprising obtaining the amino acid sequence of the desired antigen-binding agent or fragment thereof and utilizing the obtained amino acid sequence to produce additional copies of the antigen-binding agent or fragment thereof.

35. The method of claim 1, further comprising utilizing the mutant nucleic acid sequences to produce additional copies of the desired antigen-binding agent or fragment thereof.

36. The method of claim 35, wherein the additional copies of the desired antigen-binding agent or fragment thereof are produced by amplifying the mutant nucleic acid sequences to provide multiple copies of the mutant nucleic acid sequences, and expressing the multiple copies of the mutant nucleic acid sequences in a cell to produce the additional copies of the desired antigen-binding agent or fragment thereof.

37. The method of claim 35, further comprising obtaining the sequences of the mutant nucleic acid sequences and utilizing the obtained sequences to produce additional copies of the desired antigen-binding agent or fragment thereof.

38. The method of claim 37, wherein utilizing the obtained sequences to prepare additional copies of the desired antigen-binding agent or fragment thereof comprises preparing one or more nucleic acids having the obtained sequences and expressing the one or more nucleic acids in one or more cells to produce the additional copies of the desired antigen-binding agent or fragment thereof.

39. A method of identifying an antigen-binding agent that binds to an antigen of interest, which method comprises:
(a) providing a population of mammalian cells containing a first nucleic acid sequence and a set of second nucleic acid sequences,
wherein the first nucleic acid sequence encodes a first polypeptide comprising a first component of an antigen-binding agent,
wherein the set of second nucleic acid sequences comprises second nucleic acid sequences encoding second polypeptides comprising second components of antigen-binding agents, and
wherein (1) the first component and (2) a second component together form an antigen-binding agent,
(b) maintaining the population of mammalian cells under conditions wherein the first nucleic acid sequence and the set of second nucleic acid sequences are expressed to produce a set of antigen-binding agents, and
(c) identifying an antigen-binding agent that binds to the antigen of interest.

40. An antigen binding agent identified by the method of claim 1.

41. A method of producing a polypeptide which binds an antigen of interest, which method comprises inserting the antigen-binding agent of claim 40 into a polypeptide, whereby a polypeptide which binds an antigen of interest is produced.

42. A composition comprising the antigen binding agent of claim 40 and a carrier therefor.

43. A composition comprising an antigen-binding agent and a carrier therefor, wherein the antigen-binding agent is provided by a method comprising:
(a) providing a population of cells containing a first nucleic acid sequence and a set of second nucleic acid sequences,
wherein the first nucleic acid sequence encodes a first polypeptide comprising a first component of an antigen-binding agent, which first nucleic acid sequence optionally has been prepared by subjecting a nucleic acid sequence to somatic hypermutation,
wherein the set of second nucleic acid sequences comprises second nucleic acid sequences encoding second polypeptides comprising second components of antigen-binding agents, which second nucleic acid sequences optionally have been prepared by subjecting a library of nucleic acid sequences to somatic hypermutation, and
wherein (1) the first component and (2) a second component together form an antigen-binding agent, and wherein the population of cells optionally expresses activation-induced cytidine deaminase (AID),
(b) maintaining the population of cells under conditions wherein the first nucleic acid sequence and the set of second nucleic acid sequences are expressed to produce a set of antigen-binding agents,
(c) identifying an antigen-binding agent that binds to the antigen of interest, and optionally,
(d) subjecting one or both of the nucleic acid sequences encoding the identified antigen-binding agent to somatic hypermutation to provide mutant nucleic acid sequences encoding a desired antigen-binding agent that binds to the antigen of interest,
with the proviso that at least one of the first nucleic acid sequence, the second nucleic acid sequences, and/or the nucleic acid sequences encoding the identified antigen-binding agent is subjected to somatic hypermutation.

44. The method of claim 16, wherein the antibody light chain is a kappa (κ) light chain.

45. The method of claim 16, wherein the antibody light chain is a lambda (λ) light chain.


SEQUENCE LISTING (357 sequences)

SEQ ID NO: 1 - protein, 198 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSRSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 2 - protein, 198 aa, Homo sapiens [sequence omitted]

SEQ ID NO: 3 - protein, 198 aa, Canis lupus familiaris
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 4 - protein, 198 aa, Mus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 5 - protein, 198 aa, Rattus rattus
MDSLLMKQKKFLYHFKNVRWAKGRHETYLCYVVKRRDSATSFSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENHERTFKAWEGLHENSVRLTRQLRRILLPLYEVDDLRDAFRILGL

SEQ ID NO: 6 - protein, 199 aa, Bos
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 7 - protein, 198 aa, Gallus gallus domesticus
MDSLLMKRKLFLYNFKNLRWAKGRRETYLCYVVKRRDSATSCSLDFGYLRNKMGCHVEVLFLRYISAWDLDPGRCYRITWFTSWSPCYDCARHVADFLRAYPNLTLRIFTARLYFCEDRKAEPEGLRRLHRAGAQIAIMTFKDFFYCWNTFVENREKTFKAWEGLHENSVHLSRKLRRILLPLYEVDDLRDAFKTLGL

SEQ ID NO: 8 - protein, 198 aa, Sus
MDSLLMKQRQFLYQFKNVRWAKGRHETYLCYVVKRRDSATSFSLDFGHLRNKSGCHVELLFLRYISDWDLDPGRCYRVTWFTSWSPCYDCARHVANFLRGNPNLSLRIFTARLYFCDGYKAEPEGLRRLHRAGVQIAIMTFKDYFYCWNTFVENRERSFKAWEGLHENSVRLTRQLRRILLPLYEVDDLRDAFRTLGL

SEQ ID NO: 9 - protein, 198 aa, Pan troglodytes
MDSLLMNRKKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 10 - protein, 198 aa, Macaca
MDSLLMNRRKFLYHFKNVRWAKGRHETYLCYVVKRRDSATSFSLDFGHLRNKSGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENRERTFKAWEGLHENSVRLSRQLRRILLPLYEVDDLRDAFRTLGL

SEQ ID NO: 11 - protein, 239 aa, Equus
MSEVLTTRPAVPDAVPPVPNLLIHTAQEENDLCSFIFLPPLIHSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENRERTFKAWEGLHENSVRLSRQLRRILLPLYEVDDLRDAFRTLGL

SEQ ID NO: 12 - protein, 201 aa, Xenopus
MTMDSMLLKRNKFIYHYKNLRWARGRHETYLCYIVKRRYSSVSCALDFGYLRNRNGCHAEMLFLRYLSIWVGHDPHRNYRVTWFSSWSPCYDCAKRTLEFLKGHPNFSLRIFSARLYFCEERNAEPEGLRKLQKAGVRLSVMSYKDYFYCWNTFVETRESGFEAWDGLHENSVRLARKLRRILQPPYDMEDLREVFVLLGL

SEQ ID NO: 13 - protein, 204 aa, Fugu
MLLPRKKFIYHYKNVRWARGRHETYLCFVVKRRVGPDTLTFDFGHLRNRSGCHVELLFLRYLGALCPGLWGYGAAGEKRLSYSVTWFCSWSPCVNCSIQLCQFLNNTPNLRLRIFVSRLYFCDLEDSLEREGLRMLTKAGVRISVMSYKDYFYCWQKFVDCKKSNFKAWEELHQNSVRLTRKLNRILQAWDLEDLRDALKLLGF

SEQ ID NO: 14 - protein, 210 aa, Danio rerio
MICKLDSVLMTQKKFIFHYKNVRWARGRHETYLCFVVKRRIGPDSLSFDFGHLRNRSGCHVELLFLRHLGALCPGLSASSVDGARLCYSVTWFCSWSPCSKCAQQLAHFLSQTPNLRLRIFVSRLYFCDEEDSVEREGLRHLKRAGVQISVMTYKDFFYCWQTFVARRERSFKAWDGLHENSVRLVRKLNRILQPCETEDLRDVFALLGL

SEQ ID NO: 15 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRT

SEQ ID NO: 16 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSTPLTFGGGTKVEIKRT

SEQ ID NO: 17 - protein, 130 aa, Artificial Sequence; identical to SEQ ID NO: 16
  Synthetic

SEQ ID NO: 18 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYGRSPLTFGGGTKVEIKRT

SEQ ID NO: 19 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSNPITFGQGTRLEIQRT

SEQ ID NO: 20 - protein, 130 aa, Artificial Sequence; identical to SEQ ID NO: 19
  Synthetic

SEQ ID NO: 21 - protein, 130 aa, Artificial Sequence; identical to SEQ ID NO: 15
  Synthetic

SEQ ID NO: 22 - protein, 130 aa, Artificial Sequence; identical to SEQ ID NO: 16
  Synthetic

SEQ ID NO: 23 - protein, 132 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYDNVPSFTFGPGTKVDIKRT

SEQ ID NO: 24 - protein, 130 aa, Artificial Sequence; identical to SEQ ID NO: 15
  Synthetic

SEQ ID NO: 25 - protein, 131 aa, Artificial Sequence; identical to SEQ ID NO: 18
  Synthetic

SEQ ID NO: 26 - protein, 130 aa, Artificial Sequence; identical to SEQ ID NO: 15
  Synthetic

SEQ ID NO: 27 - protein, 130 aa, Artificial Sequence; identical to SEQ ID NO: 16
  Synthetic

SEQ ID NO: 28 - protein, 131 aa, Artificial Sequence; identical to SEQ ID NO: 18
  Synthetic

SEQ ID NO: 29 - protein, 130 aa, Artificial Sequence; identical to SEQ ID NO: 15
  Synthetic

SEQ ID NO: 30 - protein, 130 aa, Artificial Sequence; identical to SEQ ID NO: 16
  Synthetic

SEQ ID NO: 31 - protein, 130 aa, Artificial Sequence; identical to SEQ ID NO: 15
  Synthetic

SEQ ID NO: 32 - protein, 131 aa, Artificial Sequence; identical to SEQ ID NO: 18
  Synthetic

SEQ ID NO: 33 - protein, 131 aa, Artificial Sequence; identical to SEQ ID NO: 18
  Synthetic

SEQ ID NO: 34 - protein, 130 aa, Artificial Sequence; identical to SEQ ID NO: 15
  Synthetic

SEQ ID NO: 35 - protein, 130 aa, Artificial Sequence; identical to SEQ ID NO: 15
  Synthetic

SEQ ID NO: 36 - protein, 130 aa, Artificial Sequence; identical to SEQ ID NO: 16
  Synthetic

SEQ ID NO: 37 - protein, 130 aa, Artificial Sequence; identical to SEQ ID NO: 19
  Synthetic

SEQ ID NO: 38 - protein, 130 aa, Artificial Sequence; identical to SEQ ID NO: 15
  Synthetic

SEQ ID NO: 39 - protein, 130 aa, Artificial Sequence; identical to SEQ ID NO: 19
  Synthetic

SEQ ID NO: 40 - protein, 130 aa, Artificial Sequence; identical to SEQ ID NO: 16
  Synthetic

SEQ ID NO: 41 - protein, 130 aa, Artificial Sequence; identical to SEQ ID NO: 15
  Synthetic

SEQ ID NO: 42 - protein, 130 aa, Artificial Sequence; identical to SEQ ID NO: 15
  Synthetic

SEQ ID NO: 43 - protein, 130 aa, Artificial Sequence; identical to SEQ ID NO: 15
  Synthetic

SEQ ID NO: 44 - protein, 130 aa, Artificial Sequence; identical to SEQ ID NO: 15
  Synthetic

SEQ ID NO: 45 - protein, 131 aa, Artificial Sequence; identical to SEQ ID NO: 18
  Synthetic

SEQ ID NO: 46 - protein, 131 aa, Artificial Sequence; identical to SEQ ID NO: 18
  Synthetic

SEQ ID NO: 47 - protein, 130 aa, Artificial Sequence; identical to SEQ ID NO: 15
  Synthetic

SEQ ID NO: 48 - protein, 130 aa, Artificial Sequence; identical to SEQ ID NO: 16
  Synthetic

SEQ ID NO: 49 - protein, 130 aa, Artificial Sequence; identical to SEQ ID NO: 15
  Synthetic

SEQ ID NO: 50 - protein, 130 aa, Artificial Sequence; identical to SEQ ID NO: 16
  Synthetic

SEQ ID NO: 51 - protein, 130 aa, Artificial Sequence; identical to SEQ ID NO: 15
  Synthetic

SEQ ID NO: 52 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSTPCSFGQGTKLEIKRT

SEQ ID NO: 53 - protein, 130 aa, Artificial Sequence; identical to SEQ ID NO: 15
  Synthetic

SEQ ID NO: 54 - protein, 130 aa, Artificial Sequence; identical to SEQ ID NO: 15
  Synthetic

SEQ ID NO: 55 - protein, 130 aa, Artificial Sequence; identical to SEQ ID NO: 15
  Synthetic

SEQ ID NO: 56 - protein, 130 aa, Artificial Sequence
  Synthetic
METPAQLLFLLLLWLPDTTGEIVLTQSPGTLSLSPGERATLSCRASQSVSSSYLACYQQKPGQAPRLLIYGASSRATGIPDRFSGSGSGTDFTLTISRLEPEDFAVYYCQQYGSSPNTFGQGTKLEIKRT

SEQ ID NO: 57 - protein, 131 aa, Artificial Sequence; identical to SEQ ID NO: 18
  Synthetic

SEQ ID NO: 58 - protein, 130 aa, Artificial Sequence; identical to SEQ ID NO: 15
  Synthetic

SEQ ID NO: 59 - protein, 130 aa, Artificial Sequence; identical to SEQ ID NO: 15
  Synthetic

SEQ ID NO: 60 - protein, 130 aa, Artificial Sequence; identical to SEQ ID NO: 15
  Synthetic

SEQ ID NO: 61 - protein, 131 aa, Artificial Sequence; identical to SEQ ID NO: 18
  Synthetic

SEQ ID NO: 62 - protein, 131 aa, Artificial Sequence; identical to SEQ ID NO: 18
  Synthetic

SEQ ID NO: 63 - protein, 131 aa, Artificial Sequence; identical to SEQ ID NO: 18
  Synthetic

SEQ ID NO: 64 - protein, 130 aa, Artificial Sequence
  Synthetic
METPAQLLFLPLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSNPITFGQGTRLEIQRT

SEQ ID NO: 65 - protein, 130 aa, Artificial Sequence; identical to SEQ ID NO: 15
  Synthetic

SEQ ID NO: 66 - protein, 130 aa, Artificial Sequence; identical to SEQ ID NO: 15
  Synthetic

SEQ ID NO: 67 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPNTFGQGTKLEIKRT

SEQ ID NO: 68 - protein, 130 aa, Artificial Sequence; identical to SEQ ID NO: 15
  Synthetic

SEQ ID NO: 69 - protein, 130 aa, Artificial Sequence; identical to SEQ ID NO: 15
  Synthetic

SEQ ID NO: 70 - protein, 130 aa, Artificial Sequence; identical to SEQ ID NO: 15
  Synthetic

SEQ ID NO: 71 - protein, 120 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPNTFG

SEQ ID NO: 72 - protein, 130 aa, Artificial Sequence; identical to SEQ ID NO: 15
  Synthetic

SEQ ID NO: 73 - protein, 130 aa, Artificial Sequence; identical to SEQ ID NO: 15
  Synthetic

SEQ ID NO: 74 - protein, 130 aa, Artificial Sequence; identical to SEQ ID NO: 15
  Synthetic

SEQ ID NO: 75 - protein, 130 aa, Artificial Sequence; identical to SEQ ID NO: 15
  Synthetic

SEQ ID NO: 76 - protein, 130 aa, Artificial Sequence; identical to SEQ ID NO: 15
  Synthetic

SEQ ID NO: 77 - protein, 130 aa, Artificial Sequence; identical to SEQ ID NO: 15
  Synthetic

SEQ ID NO: 78 - protein, 130 aa, Artificial Sequence; identical to SEQ ID NO: 15
  Synthetic

SEQ ID NO: 79 - protein, 130 aa, Artificial Sequence
  Synthetic
METPAQLLFLLLLWFPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSTPLTFGGGTKVEIKRT

SEQ ID NO: 80 - protein, 130 aa, Artificial Sequence; identical to SEQ ID NO: 15
  Synthetic

SEQ ID NO: 81 - protein, 130 aa, Artificial Sequence; identical to SEQ ID NO: 15
  Synthetic

SEQ ID NO: 82 - protein, 130 aa, Artificial Sequence; identical to SEQ ID NO: 15
  Synthetic

SEQ ID NO: 83 - protein, 130 aa, Artificial Sequence; identical to SEQ ID NO: 15
  Synthetic

SEQ ID NO: 84 - protein, 130 aa, Artificial Sequence; identical to SEQ ID NO: 15
  Synthetic

SEQ ID NO: 85 - protein, 130 aa, Artificial Sequence; identical to SEQ ID NO: 16
  Synthetic

SEQ ID NO: 86 - protein, 130 aa, Artificial Sequence; identical to SEQ ID NO: 15
  Synthetic

SEQ ID NO: 87 - protein, 130 aa, Artificial Sequence; identical to SEQ ID NO: 15
  Synthetic

SEQ ID NO: 88 - protein, 130 aa, Artificial Sequence; identical to SEQ ID NO: 15
  Synthetic

SEQ ID NO: 89 - protein, 130 aa, Artificial Sequence; identical to SEQ ID NO: 15
  Synthetic

SEQ ID NO: 90 - protein, 130 aa, Artificial Sequence; identical to SEQ ID NO: 15
  Synthetic

SEQ ID NO: 91 - protein, 130 aa, Artificial Sequence; identical to SEQ ID NO: 15
  Synthetic

SEQ ID NO: 92 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGVSSRATGIPDRFSGSGSGTDFTLTISRLEPEDFAVYYCQQYYSTPLTFGGGTKVEIKRT

SEQ ID NO: 93 - protein, 130 aa, Artificial Sequence; identical to SEQ ID NO: 15
  Synthetic

SEQ ID NO: 94 - protein, 130 aa, Artificial Sequence; identical to SEQ ID NO: 15
  Synthetic

SEQ ID NO: 95 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSSPLTFGGGTKVEIKRT

SEQ ID NO: 96 - protein, 130 aa, Artificial Sequence; identical to SEQ ID NO: 15
  Synthetic

SEQ ID NO: 97 - protein, 130 aa, Artificial Sequence; identical to SEQ ID NO: 15
  Synthetic

SEQ ID NO: 98 - protein, 130 aa, Artificial Sequence; identical to SEQ ID NO: 15
  Synthetic

SEQ ID NO: 99 - protein, 130 aa, Artificial Sequence; identical to SEQ ID NO: 15
  Synthetic

SEQ ID NO: 100 - protein, 130 aa, Artificial Sequence; identical to SEQ ID NO: 15
  Synthetic

SEQ ID NO: 101 - protein, 130 aa, Artificial Sequence; identical to SEQ ID NO: 15
  Synthetic

SEQ ID NO: 102 - protein, 130 aa, Artificial Sequence; identical to SEQ ID NO: 16
  Synthetic

SEQ ID NO: 103 - protein, 130 aa, Artificial Sequence; identical to SEQ ID NO: 16
  Synthetic

SEQ ID NO: 104 - protein, 130 aa, Artificial Sequence; identical to SEQ ID NO: 15
  Synthetic

SEQ ID NO: 105 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGADFTLTISRLEPEDFAVYYCQQYGSSPNTFGQGTKLEIKRT

SEQ ID NO: 106 - protein, 130 aa, Artificial Sequence; identical to SEQ ID NO: 15
  Synthetic

SEQ ID NO: 107 - protein, 130 aa, Artificial Sequence; identical to SEQ ID NO: 16
  Synthetic

SEQ ID NO: 108 - protein, 130 aa, Artificial Sequence; identical to SEQ ID NO: 16
  Synthetic

SEQ ID NO: 109 - protein, 130 aa, Artificial Sequence; identical to SEQ ID NO: 15
  Synthetic

SEQ ID NO: 110 - protein, 130 aa, Artificial Sequence; identical to SEQ ID NO: 15
  Synthetic

SEQ ID NO: 111 - protein, 130 aa, Artificial Sequence; identical to SEQ ID NO: 15
  Synthetic

SEQ ID NO: 112 - protein, 130 aa, Artificial Sequence; identical to SEQ ID NO: 15
  Synthetic

SEQ ID NO: 113 - protein, 130 aa, Artificial Sequence; identical to SEQ ID NO: 15
  Synthetic

SEQ ID NO: 114 - protein, 130 aa, Artificial Sequence; identical to SEQ ID NO: 16
  Synthetic

SEQ ID NO: 115 - protein, 130 aa, Artificial Sequence; identical to SEQ ID NO: 16
  Synthetic

SEQ ID NO: 116 - protein, 130 aa, Artificial Sequence
  Synthetic
METPAQLLFLLLLWLPDTTGEIVLTQFPGTLFLFPGERATLFCRASQSVSSSYLAWYQQKPGQAPRLLIYGASSRATGIPDRFSGSGFGTDFTLTISRLEPEDFAVYYCQQYYSTPLTFGGGTKVEIKRT

SEQ ID NO: 117 - protein, 131 aa, Artificial Sequence; identical to SEQ ID NO: 18
  Synthetic

SEQ ID NO: 118 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSSLLTFGGGTKVEINRT

SEQ ID NO: 119 - protein, 130 aa, Artificial Sequence; identical to SEQ ID NO: 16
  Synthetic

SEQ ID NO: 120 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQSYSTLYTFGQGTKVEIKRT

SEQ ID NO: 121 - protein, 131 aa, Artificial Sequence; identical to SEQ ID NO: 18
  Synthetic

SEQ ID NO: 122 - protein, 131 aa, Artificial Sequence; identical to SEQ ID NO: 18
  Synthetic

SEQ ID NO: 123 - protein, 130 aa, Artificial Sequence; identical to SEQ ID NO: 19
  Synthetic

SEQ ID NO: 124 - protein, 130 aa, Artificial Sequence; identical to SEQ ID NO: 15
  Synthetic

SEQ ID NO: 125 - protein, 131 aa, Artificial Sequence; identical to SEQ ID NO: 18
  Synthetic

SEQ ID NO: 126 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIRRT

SEQ ID NO: 127 - protein, 130 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYYSTPFFGQGTKLEIKRT

SEQ ID NO: 128 - protein, 130 aa, Artificial Sequence; identical to SEQ ID NO: 15
  Synthetic

SEQ ID NO: 129 - protein, 131 aa, Artificial Sequence; identical to SEQ ID NO: 18
  Synthetic

SEQ ID NO: 130 - protein, 130 aa, Artificial Sequence; identical to SEQ ID NO: 120
  Synthetic

SEQ ID NO: 131 - protein, 130 aa, Artificial Sequence; identical to SEQ ID NO: 15
  Synthetic

SEQ ID NO: 132 - protein, 131 aa, Artificial Sequence; identical to SEQ ID NO: 18
  Synthetic

SEQ ID NO: 133 - protein, 130 aa, Artificial Sequence; identical to SEQ ID NO: 19
  Synthetic

SEQ ID NO: 134 - protein, 131 aa, Artificial Sequence; identical to SEQ ID NO: 18
  Synthetic

SEQ ID NO: 135 - protein, 130 aa, Artificial Sequence; identical to SEQ ID NO: 19
  Synthetic

SEQ ID NO: 136 - protein, 130 aa, Artificial Sequence; identical to SEQ ID NO: 52
  Synthetic

SEQ ID NO: 137 - protein, 130 aa, Artificial Sequence; identical to SEQ ID NO: 15
  Synthetic

SEQ ID NO: 138 - protein, 130 aa, Artificial Sequence; identical to SEQ ID NO: 15
  Synthetic

SEQ ID NO: 139 - protein, 133 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYGSSPPPTTFGQGTKVEIKRT

SEQ ID NO: 140 - protein, 130 aa, Artificial Sequence; identical to SEQ ID NO: 15
  Synthetic

SEQ ID NO: 141 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPLTFGGGTKVEIKRT

SEQ ID NO: 142 - protein, 131 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRRSFGQGTKLEIKRT

SEQ ID NO: 143 - protein, 130 aa, Artificial Sequence; identical to SEQ ID NO: 15
  Synthetic

SEQ ID NO: 144 - protein, 131 aa, Artificial Sequence; identical to SEQ ID NO: 18
  Synthetic

SEQ ID NO: 145 - protein, 131 aa, Artificial Sequence; identical to SEQ ID NO: 18
  Synthetic

SEQ ID NO: 146 - protein, 130 aa, Artificial Sequence; identical to SEQ ID NO: 15
  Synthetic

SEQ ID NO: 147 - protein, 130 aa, Artificial Sequence; identical to SEQ ID NO: 52
  Synthetic

SEQ ID NO: 148 - protein, 130 aa, Artificial Sequence; identical to SEQ ID NO: 19
  Synthetic

SEQ ID NO: 149 - protein, 130 aa, Artificial Sequence; identical to SEQ ID NO: 15
  Synthetic

SEQ ID NO: 150 - protein, 131 aa, Artificial Sequence; identical to SEQ ID NO: 18
  Synthetic

SEQ ID NO: 151 - protein, 130 aa, Artificial Sequence; identical to SEQ ID NO: 19
  Synthetic

SEQ ID NO: 152 - protein, 130 aa, Artificial Sequence; identical to SEQ ID NO: 19
  Synthetic

SEQ ID NO: 153 - protein, 130 aa, Artificial Sequence
  Synthetic
METPAQLLFFLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRT

SEQ ID NO: 154 - protein, 130 aa, Artificial Sequence; identical to SEQ ID NO: 15
  Synthetic

SEQ ID NO: 155 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCNFGQGTRLEMKRT

SEQ ID NO: 156 - protein, 131 aa, Artificial Sequence; identical to SEQ ID NO: 18
  Synthetic

SEQ ID NO: 157 - protein, 130 aa, Artificial Sequence; identical to SEQ ID NO: 19
  Synthetic

SEQ ID NO: 158 - protein, 130 aa, Artificial Sequence; identical to SEQ ID NO: 15
  Synthetic

SEQ ID NO: 159 - protein, 130 aa, Artificial Sequence; identical to SEQ ID NO: 155
  Synthetic

SEQ ID NO: 160 - protein, 130 aa, Artificial Sequence
  Synthetic
METPAQLLFLLLLWLPDTTGEIVLTQSPGTLSLSPGERATLSCWASQSVSSSYLAWYQQKPGQAPRLLIYGASSRATGIPDRFSGSGSGTDFTLTISRLEPEDFAVYYCQQYYSTPLTFGQGTRLEIKRT

SEQ ID NO: 161 - protein, 130 aa, Artificial Sequence; identical to SEQ ID NO: 19
  Synthetic

SEQ ID NO: 162 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISRLEPEDFAVYYCQQYGSSPLTFGGGTKVEIKRT

SEQ ID NO: 163 - protein, 130 aa, Artificial Sequence; identical to SEQ ID NO: 19
  Synthetic

SEQ ID NO: 164 - protein, 131 aa, Artificial Sequence; identical to SEQ ID NO: 18
  Synthetic

SEQ ID NO: 165 - protein, 131 aa, Artificial Sequence
  Synthetic
MDMRVPAQLLGLLLLWLSGARCDIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIKRT

SEQ ID NO: 166 - protein, 131 aa, Artificial Sequence; identical to SEQ ID NO: 165
  Synthetic

SEQ ID NO: 167 - protein, 131 aa, Artificial Sequence; identical to SEQ ID NO: 165
  Synthetic

SEQ ID NO: 168 - protein, 131 aa, Artificial Sequence; identical to SEQ ID NO: 165
  Synthetic

SEQ ID NO: 169 - protein, 131 aa, Artificial Sequence; identical to SEQ ID NO: 165
  Synthetic

SEQ ID NO: 170 - protein, 131 aa, Artificial Sequence; identical to SEQ ID NO: 165
  Synthetic

SEQ ID NO: 171 - protein, 131 aa, Artificial Sequence; identical to SEQ ID NO: 165
  Synthetic

SEQ ID NO: 172 - protein, 131 aa, Artificial Sequence; identical to SEQ ID NO: 165
  Synthetic

SEQ ID NO: 173 - protein, 131 aa, Artificial Sequence; identical to SEQ ID NO: 165
  Synthetic

SEQ ID NO: 174 - protein, 131 aa, Artificial Sequence; identical to SEQ ID NO: 165
  Synthetic

SEQ ID NO: 175 - protein, 131 aa, Artificial Sequence; identical to SEQ ID NO: 165
  Synthetic

SEQ ID NO: 176 - protein, 131 aa, Artificial Sequence; identical to SEQ ID NO: 165
  Synthetic

SEQ ID NO: 177 - protein, 131 aa, Artificial Sequence; identical to SEQ ID NO: 165
  Synthetic

SEQ ID NO: 178 - protein, 131 aa, Artificial Sequence; identical to SEQ ID NO: 165
  Synthetic

SEQ ID NO: 179 - protein, 131 aa, Artificial Sequence; identical to SEQ ID NO: 165
  Synthetic

SEQ ID NO: 180 - protein, 131 aa, Artificial Sequence; identical to SEQ ID NO: 165
  Synthetic

SEQ ID NO: 181 - protein, 131 aa, Artificial Sequence; identical to SEQ ID NO: 165
  Synthetic

SEQ ID NO: 182 - protein, 131 aa, Artificial Sequence; identical to SEQ ID NO: 165
  Synthetic

SEQ ID NO: 183 - protein, 131 aa, Artificial Sequence; identical to SEQ ID NO: 165
  Synthetic

SEQ ID NO: 184 - protein, 131 aa, Artificial Sequence; identical to SEQ ID NO: 165
  Synthetic

SEQ ID NO: 185 - protein, 131 aa, Artificial Sequence; identical to SEQ ID NO: 165
  Synthetic

SEQ ID NO: 186 - protein, 131 aa, Artificial Sequence; identical to SEQ ID NO: 165
  Synthetic

SEQ ID NO: 187 - protein, 131 aa, Artificial Sequence; identical to SEQ ID NO: 165
  Synthetic

SEQ ID NO: 188 - protein, 131 aa, Artificial Sequence; identical to SEQ ID NO: 165
  Synthetic

SEQ ID NO: 189 - protein, 131 aa, Artificial Sequence; identical to SEQ ID NO: 165
  Synthetic

SEQ ID NO: 190 - protein, 131 aa, Artificial Sequence; identical to SEQ ID NO: 165
  Synthetic

SEQ ID NO: 191 - protein, 131 aa, Artificial Sequence; identical to SEQ ID NO: 165
  Synthetic

SEQ ID NO: 192 - protein, 131 aa, Artificial Sequence; identical to SEQ ID NO: 165
  Synthetic

SEQ ID NO: 193 - protein, 131 aa, Artificial Sequence; identical to SEQ ID NO: 165
  Synthetic

SEQ ID NO: 194 - protein, 131 aa, Artificial Sequence; identical to SEQ ID NO: 165
  Synthetic

SEQ ID NO: 195 - protein, 131 aa, Artificial Sequence; identical to SEQ ID NO: 165
  Synthetic

SEQ ID NO: 196 - protein, 131 aa, Artificial Sequence; identical to SEQ ID NO: 165
  Synthetic

SEQ ID NO: 197 - protein, 131 aa, Artificial Sequence; identical to SEQ ID NO: 165
  Synthetic

SEQ ID NO: 198 - protein, 131 aa, Artificial Sequence; identical to SEQ ID NO: 165
  Synthetic

SEQ ID NO: 199 - protein, 131 aa, Artificial Sequence; identical to SEQ ID NO: 165
  Synthetic

SEQ ID NO: 200 - protein, 131 aa, Artificial Sequence; identical to SEQ ID NO: 165
  Synthetic

SEQ ID NO: 201 - protein, 130 aa, Artificial Sequence; identical to SEQ ID NO: 15
  Synthetic

SEQ ID NO: 202 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRT

SEQ ID NO: 203 - protein, 130 aa, Artificial Sequence; identical to SEQ ID NO: 202
  Synthetic

SEQ ID NO: 204 - protein, 130 aa, Artificial Sequence; identical to SEQ ID NO: 202
  Synthetic

SEQ ID NO: 205 - protein, 130 aa, Artificial Sequence; identical to SEQ ID NO: 202
  Synthetic

SEQ ID NO: 206 - protein, 130 aa, Artificial Sequence; identical to SEQ ID NO: 202
  Synthetic

SEQ ID NO: 207 - protein, 130 aa, Artificial Sequence
  Synthetic
METPAQLLFLLLLWLPDTTGEIVLTQSPGTLSLSPGERATLSCRASQSVSSSYLAWHQQKPGQAPRLLIYGASSRATGIPDRFSGSGSGTDFTLTISRLEPEDFAVYYCQQYGSSPCTFGQGTRLEIKRT

SEQ ID NO: 208 - protein, 130 aa, Artificial Sequence; identical to SEQ ID NO: 202
  Synthetic

SEQ ID NO: 209 - protein, 130 aa, Artificial Sequence; identical to SEQ ID NO: 202
  Synthetic

SEQ ID NO: 210 - protein, 130 aa, Artificial Sequence; identical to SEQ ID NO: 202
  Synthetic

SEQ ID NO: 211 - protein, 130 aa, Artificial Sequence; identical to SEQ ID NO: 202
  Synthetic

SEQ ID NO: 212 - protein, 130 aa, Artificial Sequence; identical to SEQ ID NO: 202
  Synthetic

SEQ ID NO: 213 - protein, 130 aa, Artificial Sequence; identical to SEQ ID NO: 202
  Synthetic

SEQ ID NO: 214 - protein, 130 aa, Artificial Sequence; identical to SEQ ID NO: 202
  Synthetic

SEQ ID NO: 215 - protein, 130 aa, Artificial Sequence; identical to SEQ ID NO: 202
  Synthetic

SEQ ID NO: 216 - protein, 130 aa, Artificial Sequence; identical to SEQ ID NO: 202
  Synthetic

SEQ ID NO: 217 - protein, 130 aa, Artificial Sequence; identical to SEQ ID NO: 202
  Synthetic

SEQ ID NO: 218 - protein, 130 aa, Artificial Sequence; identical to SEQ ID NO: 202
  Synthetic

SEQ ID NO: 219 - protein, 130 aa, Artificial Sequence; identical to SEQ ID NO: 202
  Synthetic

SEQ ID NO: 220 - protein, 130 aa, Artificial Sequence; identical to SEQ ID NO: 202
  Synthetic

SEQ ID NO: 221 - protein, 130 aa, Artificial Sequence; identical to SEQ ID NO: 202
  Synthetic

SEQ ID NO: 222 - protein, 130 aa, Artificial Sequence; identical to SEQ ID NO: 202
  Synthetic

SEQ ID NO: 223 - protein, 130 aa, Artificial Sequence; identical to SEQ ID NO: 202
  Synthetic

SEQ ID NO: 224 - protein, 130 aa, Artificial Sequence; identical to SEQ ID NO: 202
  Synthetic

SEQ ID NO: 225 - protein, 130 aa, Artificial Sequence; identical to SEQ ID NO: 202
  Synthetic

SEQ ID NO: 226 - protein, 130 aa, Artificial Sequence; identical to SEQ ID NO: 202
  Synthetic

SEQ ID NO: 227 - protein, 130 aa, Artificial Sequence; identical to SEQ ID NO: 202
  Synthetic

SEQ ID NO: 228 - protein, 130 aa, Artificial Sequence; identical to SEQ ID NO: 202
  Synthetic

SEQ ID NO: 229 - protein, 130 aa, Artificial Sequence; identical to SEQ ID NO: 202
  Synthetic

SEQ ID NO: 230 - protein, 130 aa, Artificial Sequence; identical to SEQ ID NO: 202
  Synthetic

SEQ ID NO: 231 - protein, 130 aa, Artificial Sequence; identical to SEQ ID NO: 202
  Synthetic

SEQ ID NO: 232 - protein, 130 aa, Artificial Sequence; identical to SEQ ID NO: 202
  Synthetic

SEQ ID NO: 233 - protein, 130 aa, Artificial Sequence
  Synthetic
METPAQLLFLPLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCTFGQGTRLEIKRT

SEQ ID NO: 234 - protein, 130 aa, Artificial Sequence; identical to SEQ ID NO: 202
  Synthetic

SEQ ID NO: 235 - protein, 130 aa, Artificial Sequence; identical to SEQ ID NO: 202
  Synthetic

SEQ ID NO: 236 - protein, 130 aa, Artificial Sequence; identical to SEQ ID NO: 202
  Synthetic

SEQ ID NO: 237 - protein, 130 aa, Artificial Sequence; identical to SEQ ID NO: 202
  Synthetic

SEQ ID NO: 238 - protein, 130 aa, Artificial Sequence; identical to SEQ ID NO: 202
  Synthetic

SEQ ID NO: 239 - protein, 130 aa, Artificial Sequence; identical to SEQ ID NO: 202
  Synthetic

SEQ ID NO: 240 - protein, 130 aa, Artificial Sequence; identical to SEQ ID NO: 202
  Synthetic

SEQ ID NO: 241 - protein, 130 aa, Artificial Sequence; identical to SEQ ID NO: 202
  Synthetic

SEQ ID NO: 242 - protein, 130 aa, Artificial Sequence; identical to SEQ ID NO: 202
  Synthetic

SEQ ID NO: 243 - protein, 130 aa, Artificial Sequence; identical to SEQ ID NO: 202
  Synthetic

SEQ ID NO: 244 - protein, 131 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPYTFGQGTKLEIKRT

SEQ ID NO: 245 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPVTFGQGTRLEIKGT

SEQ ID NO: 246 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYDNLPLTFGGGTKVEIKRT

SEQ ID NO: 247 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCSFGQGTKLEIKRT

SEQ ID NO: 248 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPTTFGQGTKVEIKRT

SEQ ID NO: 249 - protein, 130 aa, Artificial Sequence; identical to SEQ ID NO: 247
  Synthetic

SEQ ID NO: 250 - protein, 131 aa, Artificial Sequence
  Synthetic
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFAVYYCQQYGSSPQTFGQGTKLEIKRT

SEQ ID NO: 251 - protein, 131 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCMQGTHWPPWTFGQGTKVEIKRT

SEQ ID NO: 252 - protein, 130 aa, Artificial Sequence; identical to SEQ ID NO: 15
  Synthetic

SEQ ID NO: 253 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSSLLTFGGGTKVEIKRT

SEQ ID NO: 254 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPQTFGQGTKVEIKRT

SEQ ID NO: 255 - protein, 130 aa, Artificial Sequence; identical to SEQ ID NO: 15
  Synthetic

SEQ ID NO: 256 - protein, 130 aa, Artificial Sequence; identical to SEQ ID NO: 15
  Synthetic

SEQ ID NO: 257 - protein, 130 aa, Artificial Sequence; identical to SEQ ID NO: 245
  Synthetic

SEQ ID NO: 258 - protein, 134 aa, Artificial Sequence
  Synthetic
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTIGSLQAEDVAVYYCMQGTHWLTFGGGTKVEIKRT

SEQ ID NO: 259 - protein, 130 aa, Artificial Sequence; identical to SEQ ID NO: 248
  Synthetic

SEQ ID NO: 260 - protein, 131 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDLTLTISRLEPEDFAVYYCQQYGSSPPYTFGQGTKLEIKRT

SEQ ID NO: 261 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYDNSLLTFGGGTKVEIKRT

SEQ ID NO: 262 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPCNFGQGTKLEIKRT

SEQ ID NO: 263 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSTPPTFGGGTKVEIKRT

SEQ ID NO: 264 - protein, 130 aa, Artificial Sequence; identical to SEQ ID NO: 248
  Synthetic

SEQ ID NO: 265 - protein, 130 aa, Artificial Sequence; identical to SEQ ID NO: 245
  Synthetic

SEQ ID NO: 266 - protein, 135 aa, Artificial Sequence
  Synthetic
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYGSSPLTFGGGTKVEIKRT

SEQ ID NO: 267 - protein, 130 aa, Artificial Sequence; identical to SEQ ID NO: 253
  Synthetic

SEQ ID NO: 268 - protein, 130 aa, Artificial Sequence; identical to SEQ ID NO: 247
  Synthetic

SEQ ID NO: 269 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPVTFGQGTKVEIKRT

SEQ ID NO: 270 - protein, 130 aa, Artificial Sequence; identical to SEQ ID NO: 245
  Synthetic

SEQ ID NO: 271 - protein, 130 aa, Artificial Sequence; identical to SEQ ID NO: 247
  Synthetic

SEQ ID NO: 272 - protein, 130 aa, Artificial Sequence; identical to SEQ ID NO: 245
  Synthetic

SEQ ID NO: 273 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSTLNTFGQGTKLEIKRT

SEQ ID NO: 274 - protein, 130 aa, Artificial Sequence; identical to SEQ ID NO: 245
  Synthetic

SEQ ID NO: 275 - protein, 130 aa, Artificial Sequence; identical to SEQ ID NO: 245
  Synthetic

SEQ ID NO: 276 - protein, 130 aa, Artificial Sequence; identical to SEQ ID NO: 253
  Synthetic

SEQ ID NO: 277 - protein, 136 aa, Artificial Sequence
  Synthetic
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSSPPYTFGQGTKLEIRRT

SEQ ID NO: 278 - protein, 130 aa, Artificial Sequence; identical to SEQ ID NO: 247
  Synthetic

SEQ ID NO: 279 - protein, 130 aa, Artificial Sequence; identical to SEQ ID NO: 245
  Synthetic

SEQ ID NO: 280 - protein, 130 aa, Artificial Sequence; identical to SEQ ID NO: 19
  Synthetic

SEQ ID NO: 281 - protein, 130 aa, Artificial Sequence; identical to SEQ ID NO: 245
  Synthetic

SEQ ID NO: 282 - protein, 130 aa, Artificial Sequence; identical to SEQ ID NO: 247
  Synthetic

SEQ ID NO: 283 - protein, 131 aa, Artificial Sequence
  Synthetic
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFAVYYCMQGTHWALTFGGGTKVEIKRT

SEQ ID NO: 284 - protein, 130 aa, Artificial Sequence; identical to SEQ ID NO: 247
  Synthetic

SEQ ID NO: 285 - protein, 132 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYGSSPPGTFGGGTKVEIKRT

SEQ ID NO: 286 - protein, 130 aa, Artificial Sequence; identical to SEQ ID NO: 245
  Synthetic

SEQ ID NO: 287 - protein, 130 aa, Artificial Sequence; identical to SEQ ID NO: 15
  Synthetic

SEQ ID NO: 288 - protein, 130 aa, Artificial Sequence; identical to SEQ ID NO: 253
  Synthetic

SEQ ID NO: 289 - protein, 130 aa, Artificial Sequence; identical to SEQ ID NO: 247
  Synthetic

SEQ ID NO: 290 - protein, 131 aa, Artificial Sequence; identical to SEQ ID NO: 251
  Synthetic

SEQ ID NO: 291 - protein, 130 aa, Artificial Sequence; identical to SEQ ID NO: 245
  Synthetic

SEQ ID NO: 292 - protein, 130 aa, Artificial Sequence; identical to SEQ ID NO: 245
  Synthetic

SEQ ID NO: 293 - protein, 130 aa, Artificial Sequence; identical to SEQ ID NO: 247
  Synthetic

SEQ ID NO: 294 - protein, 135 aa, Artificial Sequence
  Synthetic
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYDNLPITFGQGTRLEIKRT

SEQ ID NO: 295 - protein, 130 aa, Artificial Sequence; identical to SEQ ID NO: 253
  Synthetic

SEQ ID NO: 296 - protein, 136 aa, Artificial Sequence; identical to SEQ ID NO: 277
  Synthetic

SEQ ID NO: 297 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCMQGTHWPYTFGQGTNLEIKRT

SEQ ID NO: 298 - protein, 130 aa, Artificial Sequence; identical to SEQ ID NO: 247
  Synthetic

SEQ ID NO: 299 - protein, 130 aa, Artificial Sequence; identical to SEQ ID NO: 15
  Synthetic

SEQ ID NO: 300 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYGSSPWTFGQGTKVEIKRT

SEQ ID NO: 301 - protein, 131 aa, Artificial Sequence; identical to SEQ ID NO: 300
  Synthetic

SEQ ID NO: 302 - protein, 130 aa, Artificial Sequence; identical to SEQ ID NO: 15
  Synthetic

SEQ ID NO: 303 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSSPRTFGQGTKVEIKRT

SEQ ID NO: 304 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTRLEIKRT

SEQ ID NO: 305 - protein, 131 aa, Artificial Sequence; identical to SEQ ID NO: 300
  Synthetic

SEQ ID NO: 306 - protein, 131 aa, Artificial Sequence; identical to SEQ ID NO: 300
  Synthetic

SEQ ID NO: 307 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQSYSTPRTFGGGTKVEIKRT

SEQ ID NO: 308 - protein, 131 aa, Artificial Sequence
  Synthetic
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFAVYYCQQYDKPLYTFGQGTKLEIKGT

SEQ ID NO: 309 - protein, 130 aa, Artificial Sequence; identical to SEQ ID NO: 304
  Synthetic

SEQ ID NO: 310 - protein, 130 aa, Artificial Sequence; identical to SEQ ID NO: 15
  Synthetic

SEQ ID NO: 311 - protein, 130 aa, Artificial Sequence; identical to SEQ ID NO: 304
  Synthetic

SEQ ID NO: 312 - protein, 130 aa, Artificial Sequence; identical to SEQ ID NO: 15
  Synthetic

SEQ ID NO: 313 - protein, 130 aa, Artificial Sequence; identical to SEQ ID NO: 304
  Synthetic

SEQ ID NO: 314 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLAISRLEPEDFAVYYCQQYGSSPTTFGQGTRLEIKRT

SEQ ID NO: 315 - protein, 130 aa, Artificial Sequence; identical to SEQ ID NO: 15
  Synthetic

SEQ ID NO: 316 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQSYSMPFTFGPGTQVDIKRT

SEQ ID NO: 317 - protein, 131 aa, Artificial Sequence; identical to SEQ ID NO: 300
  Synthetic

SEQ ID NO: 318 - protein, 131 aa, Artificial Sequence; identical to SEQ ID NO: 300
  Synthetic

SEQ ID NO: 319 - protein, 130 aa, Artificial Sequence; identical to SEQ ID NO: 15
  Synthetic

SEQ ID NO: 320 - protein, 130 aa, Artificial Sequence; identical to SEQ ID NO: 15
  Synthetic

SEQ ID NO: 321 - protein, 130 aa, Artificial Sequence; identical to SEQ ID NO: 15
  Synthetic

SEQ ID NO: 322 - protein, 130 aa, Artificial Sequence; identical to SEQ ID NO: 304
  Synthetic

SEQ ID NO: 323 - protein, 131 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSWPPLTCGGGTKVEIKRT

SEQ ID NO: 324 - protein, 131 aa, Artificial Sequence; identical to SEQ ID NO: 300
  Synthetic

SEQ ID NO: 325 - protein, 130 aa, Artificial Sequence; identical to SEQ ID NO: 15
  Synthetic

SEQ ID NO: 326 - protein, 131 aa, Artificial Sequence; identical to SEQ ID NO: 300
  Synthetic

SEQ ID NO: 327 - protein, 131 aa, Artificial Sequence; identical to SEQ ID NO: 300
  Synthetic

SEQ ID NO: 328 - protein, 130 aa, Artificial Sequence; identical to SEQ ID NO: 304
  Synthetic

SEQ ID NO: 329 - protein, 130 aa, Artificial Sequence; identical to SEQ ID NO: 15
  Synthetic

SEQ ID NO: 330 - protein, 131 aa, Artificial Sequence; identical to SEQ ID NO: 300
  Synthetic

SEQ ID NO: 331 - protein, 132 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYGSSRGRTFGQGTKVEIKRT

SEQ ID NO: 332 - protein, 130 aa, Artificial Sequence; identical to SEQ ID NO: 304
  Synthetic

SEQ ID NO: 333 - protein, 131 aa, Artificial Sequence; identical to SEQ ID NO: 300
  Synthetic

SEQ ID NO: 334 - protein, 132 aa, Artificial Sequence; identical to SEQ ID NO: 331
  Synthetic

SEQ ID NO: 335 - protein, 130 aa, Artificial Sequence; identical to SEQ ID NO: 304
  Synthetic

SEQ ID NO: 336 - protein, 131 aa, Artificial Sequence; identical to SEQ ID NO: 300
  Synthetic

SEQ ID NO: 337 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSTPFTFGPGTKVDIKRT

SEQ ID NO: 338 - protein, 130 aa, Artificial Sequence; identical to SEQ ID NO: 15
  Synthetic

SEQ ID NO: 339 - protein, 131 aa, Artificial Sequence; identical to SEQ ID NO: 300
  Synthetic

SEQ ID NO: 340 - protein, 131 aa, Artificial Sequence; identical to SEQ ID NO: 300
  Synthetic

SEQ ID NO: 341 - protein, 130 aa, Artificial Sequence; identical to SEQ ID NO: 304
  Synthetic

SEQ ID NO: 342 - protein, 130 aa, Artificial Sequence; identical to SEQ ID NO: 304
  Synthetic

SEQ ID NO: 343 - protein, 135 aa, Artificial Sequence
  Synthetic
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYGSSPWTFGQGTKVEIKRT

SEQ ID NO: 344 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSTLRTFGQGTKVEIKRT

SEQ ID NO: 345 - protein, 130 aa, Artificial Sequence; identical to SEQ ID NO: 15
  Synthetic

SEQ ID NO: 346 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYYSTPPTFGRGTKVEIKRT

SEQ ID NO: 347 - protein, 132 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCMQGTHWPPYTFGQGTKLEIKRT

SEQ ID NO: 348 - protein, 132 aa, Artificial Sequence; identical to SEQ ID NO: 347
  Synthetic

SEQ ID NO: 349 - protein, 135 aa, Artificial Sequence
  Synthetic
MVLQTQVFISLLLWISGAYGDIVMTQSPDSLAVSLGERATINCKSSQSVLYSSNNKNYLAWYQQKPGQPPKLLIYWASTRESGVPDRFSGSGSGTDFTLTISSLQAEDVAVYYCMQGTQTPYTFGQGTKLEIKRT

SEQ ID NO: 350 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYYSSPNTFGQGTKLEIKRT

SEQ ID NO: 351 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCMQGTHWPQTFGQGTKVEIKRT

SEQ ID NO: 352 - protein, 132 aa, Artificial Sequence
  Synthetic
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFAVYYCQQYGSSPRYTFGQGTKLEIKRT

SEQ ID NO: 353 - protein, 131 aa, Artificial Sequence
  Synthetic
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFAVYYCQQSYSIPRTFGQGTKVEIKRT

SEQ ID NO: 354 - protein, 130 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIKRT

SEQ ID NO: 355 - protein, 131 aa, Artificial Sequence
  Synthetic
MDMRVPAQLLGLLLLWLRGARCDIQMTQSPSSLSASVGDRVTITCRASQSISSYLNWYQQKPGKAPKLLIYAASSLQSGVPSRFSGSGSGTDFTLTISSLQPEDFAVYYCMQGTHWPPTFGQGTKVEIKRT

SEQ ID NO: 356 - protein, 131 aa, Artificial Sequence
  Synthetic
MDMRVPAQLLGLLQLWLSGARCDIQMTQSPSSLSASVGDRVTITCQASQDISNYLNWYQQKPGKAPKLLIYDASNLETGVPSRFSGSGSGTDFTFTISSLQPEDIAVYYCQQYYSSPRTFGQGTKVEIKRT

SEQ ID NO: 357 - protein, 129 aa, Artificial Sequence
  Synthetic
METPAQLLFLLLLWLPDTTGEIVLTQSPGTLSLSPGERATLSCRASQSVSSSYLAWYQQKPGQAPRLLIYGASSRATGIPDRFSGSGSGTDFTLTISRLEPEDFAVYYCQQYDNLLTFGGGTKVEIKRT